SEQUENCE LISTING (3 sequences)

SEQ ID NO: 1 - protein, 1210 aa, Homo sapiens [sequence omitted]

SEQ ID NO: 2 - protein, 169 aa, Homo sapiens
MTAGRRMEMLCAGRVPALLLCLGFHLLQAVLSTTVIPSCIPGESSDNCTALVQTEDNPRVAQVSITKCSSDMNGYCLHGQCIYLVDMSQNYCRCEVGYTGVRCEHFFLTVHQPLSKEYVALTVILIILFLITVVGSTYYFCRWYRNRKSKEPKKEYERVTSGDPELPQV

SEQ ID NO: 3 - protein, 252 aa, Homo sapiens
MRAPLLPPAPVVLSLLILGSGHYAAGLDLNDTYSGKREPFSGDHSADGFEVTSRSEMSSGSEISPVSEMPSSSEPSSGADYDYSEEYDNEPQIPGYIVDDSVRVEQVVKPPQNKTESENTSDKPKRKKKGGKNGKNRRNRKKKNPCNAEFQNFCIHGECKYIEHLEAVTCKCQQEYFGERCGEKSMKTHSMIDSSLSKIALAAIAAFMSAVILTAVAVITVQLRRQYVRKYEGEAEERKKLRQENGNVHAIA

(19) United States
(12) Patent Application Publication
(10) Pub. No.: US 2023/0280345 A1
(43) Pub. Date: Sep. 7, 2023

(54) PREDICTION OF RESPONSE TO EPIDERMAL GROWTH FACTOR RECEPTOR-DIRECTED THERAPIES USING EPIREGULIN AND AMPHIREGULIN

(71) Applicants: VENTANA MEDICAL SYSTEMS, INC., TUCSON, AZ (US); UNIVERSITY OF LEEDS, YORKSHIRE (GB)

(72) Inventors: ISAAC YUYU BAI, ORO VALLEY, AZ (US); JENNIFER HELEN BARRETT, LEEDS (GB); FAYE ELLIOTT, LEEDS (GB); WEN-WEI LIU, TUCSON, AZ (US); PHILIP QUIRKE, LEEDS (GB); SUSAN DIANE RICHMAN, BURLEY-IN-WHAFEDALE (GB); JENNIFER FRANCES SELIGMANN, LEEDS (GB); MATTHEW THOMAS SEYMOUR, LEEDS (GB); KANDAVEL SHANMUGAM, ORO VALLEY, AZ (US); SHALINI SINGH, TUSON, AZ (US); NICHOLAS PAUL WEST, LEEDS (GB); CHRISTOPHER JAMES MICHAEL WILLIAMS, LEEDS (GB); LIPING ZHANG, ORO VALLEY, AZ (US)

(21) Appl. No.: 18/186,765

(22) Filed: Mar. 20, 2023

Related U.S. Application Data
(63) Continuation of application No. PCT/US21/50777, filed on Sep. 17, 2021.
(60) Provisional application No. 62/706,988, filed on Sep. 22, 2020.

(51) Int. Cl. G01N 33/574; A61P 35/00; C07K 16/28; A61K 31/4745

(52) U.S. Cl. CPC G01N 33/57419; A61P 35/00; C07K 16/2863; A61K 31/4745; G01N 2333/71

(57) ABSTRACT

Methods allowing prediction of a response to anti-EGFR therapies are provided, which include histochemical or cytochemical staining methods for staining amphiregulin (AREG) or epiregulin (EREG). Scoring algorithms are provided that may include but are not limited to determining a percent tumor cell positivity for each of EREG and AREG and comparing the determined percent positivity to pre-determined cut offs. The pre-determined cut offs can be either positive cut offs (in which case patients are treated with the EGFR-directed therapy if the percentage is greater than or equal to the cut off), negative cut offs (in which case patients are not treated with the EGFR-directed therapy if the percentage is less than the cut off), or both a positive and negative cut off.

### CROSS-REFERENCE TO RELATED APPLICATIONS

The present application is a continuation of International Application No. PCT/US2021/050777, which application claims the benefit of the filing date of U.S. Provisional Patent Application No. 62/706,988 filed on Sep. 22, 2020, the disclosure of which is hereby incorporated by reference herein in its entirety.

### FIELD OF THE DISCLOSURE

The present disclosure relates to histochemical or cytochemical methods, systems, and compositions for predicting response to epidermal growth factor receptor (EGFR) directed therapies. The present disclosure is also directed to methods of analyzing histology or cytology specimens so as to predict a response to an epidermal growth factor receptor (EREG) directed therapeutic agent. More particularly, the present disclosure relates to scoring methods for use in the prediction of a response to an EGFR-directed therapeutic agent based on both the percentage of tumor cells within a sample of the tumor that is positive for amphiregulin (AREG) and the percentage of tumor cells within a sample of the tumor that is positive for EREG.

### SEQUENCE LISTING INCORPORATION-BY-REFERENCE

The contents of the electronic sequence listing (Ventana-0206US1.xml; Size: 5,386 bytes; and Date of Creation: Mar. 7, 2023) is herein incorporated by reference in its entirety.

### REFERENCE TO JOINT RESEARCH AGREEMENT

This application was made by or on behalf of one or more parties to a joint research agreement between Ventana Medical Systems, Inc. and the University of Leeds.

### BACKGROUND OF THE DISCLOSURE

About 20% of patients with colon cancer present with metastatic colorectal cancer (mCRC). More than half (50-60%) of these patients will eventually develop incurable advanced disease, which has a 5 year survival rate of approximately 12.5%. Two signaling pathways in mCRC have been the focus of therapeutic drug development: the vascular endothelial growth factor receptor (VEGFR) and the epidermal growth factor receptor (EGFR) pathways. Currently, the majority of the patients with mCRC receive cytotoxic chemotherapy combined with either EGFR or VEGF-targeted therapies. EGFR is overexpressed in about 70% of CRC cases where it is associated with poor outcome. Targeted inhibition of EGFR with monoclonal antibodies, such as cetuximab or panitumumab, was approved by FDA in 2004 and 2006 to treat patients with mCRC. Both drugs have very similar efficacy with a 10-15% response rate.

A reliable positive predictor of responsiveness to EGFR-directed therapies has been lacking for some time.

Clinical studies have demonstrated that EGFR inhibitors are the most effective in patients lacking RAS pathway mutations. Point mutations in members of the RAS signaling pathways such as KRAS, NRAS, and BRAF lead to continuous activation of downstream RAS-MAPK signaling, regardless of whether the EGFR pharmacologically inactivated. In addition to RAS and BRAF mutations, other alternative mechanisms such as cMET or EGFR amplification play a role in resistance to cetuximab or panitumumab. Mutation in PI3K or PTEN loss (which often occur with RAS or BRAF mutations) may also be associated with a lack of response. Indeed, RAS, BRAF, and PI3K mutations account for more than 60% of patients with mCRC that show de novo resistance to EGFR-targeted monoclonal antibodies. Of the 40% of patients with KRAS, NRAS, BRAF and PI3K wild type tumors (quadruple wild type patients), approximately half of these patients (only 15%) benefit from anti-EGFR therapy, and more than 20% are non-responders. See Perkins et al.

Over-expression of EGFR ligands—including the ligands epiregulin (EREG) and amphiregulin (AREG)—has been suggested as a predictor for anti-EGFR therapy. In one study of patients with mCRC, addition of anti-EGFR therapy increased survival from 5.1 to 9.8 months in patients having high EREG expression levels compared to the best supportive care alone. This result suggests that EGFR ligands expression might become a clinically useful biomarker to screen patients with mCRC for EGFR inhibitor therapy. However, PCR-based detection systems lack any spatial context, such as distribution and relative abundance of cells that express the ligands.

Immunohistochemical analysis of EGFR ligands has had mixed results. Khelwatty et al., for example, showed that co-expression of wild type EGFR and at least one of its ligands (at a cut off of >5% EGFR positive tumor cells and 2+ staining intensity for the ligand) significantly correlates for a shorter progression-free survival, and thus a lower response rate to EGFR-directed therapeutic agent. However, in their samples, EGFR staining was predominantly cytoplasmic, which led them to theorize that internalization of EGFR makes it unavailable for the EGFR therapy to assert antibody-dependent cell-mediated cytotoxicity (ADCC). They further noted that up to 40% of the patients in the study may have previously received cetuximab therapy, which may have contributed to downregulation of EGFR from the surface. Khelwatty therefore does not describe a clear correlation between expression patterns of EGFR and EGFR ligand and response to EGFR-directed therapeutics. Yoshida et al. (Journal of Cancer Research and Clinical Oncology, March 2013, Volume 139, Issue 3, pp 367-378), on the other hand, found good correlation between 4 of the 7 ligands (AREG, HB-EGF, TGFα, and EREG) and clinical response to EGFR therapies, and suggest a scoring algorithm requiring at least 2 of the ligands to score in the HIGH category. Yoshida's approach, however, runs the risk of missing patients who may only over-express one of the EGFR ligands but at high levels.

### SUMMARY OF THE DISCLOSURE

This disclosure relates generally to scoring methods for use in the prediction of response to an EGFR-directed therapeutic agent based on both the percentage of tumor cells within a sample of the tumor that is positive for AREG and the percentage of tumor cells within a sample of the tumor that is positive for EREG.

In an embodiment, a method of treating patients with a tumor is provided, the method comprising administering to the patient an EGFR-directed therapeutic agent if the tumor is either AREG HIGH or EREG HIGH, wherein the tumor is considered AREG HIGH if the tumor has been histochemically or cytochemically determined to have a percentage of AREG+ tumor cells that is greater than or equal to a first pre-determined cut off and wherein the tumor is considered EREG HIGH if the tumor has been histochemically or cytochemically determined to have a percentage of EREG+ tumor cells that is greater than or equal to a second pre-determined cut off.

In another embodiment, a method of treating patients with a tumor is provided, the method comprising administering a treatment to the patient that does not include an EGFR-directed therapeutic agent if the tumor is both AREG LOW and EREG LOW, wherein the tumor is AREG LOW if the tumor has been histochemically or cytochemically determined to have a percentage of AREG+ tumor cells that is less than a first pre-determined cut off and wherein the tumor is considered EREG LOW if the tumor has been histochemically or cytochemically determined to have a percentage of EREG+ tumor cells that is less than a second pre-determined cut off.

In another embodiment, a method of selecting patients with a tumor to receive an EGFR-directed therapeutic agent is provided, the method comprising: (a) histochemically or cytochemically staining a sample of the tumor for human AREG protein; (b) histochemically or cytochemically staining a sample of the tumor for human EREG protein; (c) quantitating a percentage of AREG+ tumor cells in the sample of the tumor and comparing the percentage to a first pre-determined cut off; and (d) quantitating a percentage of EREG+ tumor cells in the sample of the tumor and comparing the percentage to a second pre-determined cut off, wherein the patient is selected to receive the EGFR-directed therapeutic agent if either the percentage of AREG+ tumor cells is greater than or equal to the first pre-determined cut off or the percentage of EREG+ tumor cells is greater than or equal to the second pre-determined cut off.

In another embodiment, a method of selecting patients with a tumor to receive a therapy that does not include an EGFR-directed therapeutic agent is provided, the method comprising: (a) histochemically or cytochemically staining a sample of the tumor for human AREG protein; (b) histochemically or cytochemically staining a sample of the tumor for human EREG protein; (c) quantitating a percentage of AREG+ tumor cells in the sample of the tumor and comparing the percentage to a first pre-determined cut off; and (d) quantitating a percentage of EREG+ tumor cells in the sample of the tumor and comparing the percentage to a second pre-determined cut off, wherein the patient is selected to receive the EGFR-directed therapeutic agent if the percentage of AREG+ tumor cells is less than the first pre-determined cut off and the percentage of EREG+ tumor cells is less than the second pre-determined cut off.

In some embodiments, the first pre-determined cut off of the foregoing methods is in the range of 20% to 50%, such as 20%, 25%, 30%, about 33.3%, 40%, and 50%.

In some embodiments, the second pre-determined cut off of the foregoing methods is in the range of 20% to 50%, such as 20%, 25%, 30%, about 33.3%, 40%, and 50%.

In some embodiments, the first pre-determined cut off of the foregoing methods is 20% and the second pre-determined cut off of the foregoing methods is 20%.

In some embodiments, the first pre-determined cut off of the foregoing methods is 25% and the second pre-determined cut off of the foregoing methods is 25%.

In some embodiments, the first pre-determined cut off of the foregoing methods is 30% and the second pre-determined cut off of the foregoing methods is 30%.

In some embodiments, the first pre-determined cut off of the foregoing methods is 33.3% and the second pre-determined cut off of the foregoing methods is 33.3%.

In some embodiments, the first pre-determined cut off of the foregoing methods is 40% and the second pre-determined cut off of the foregoing methods is 40%.

In some embodiments, the first pre-determined cut off of the foregoing methods is 50% and the second pre-determined cut off of the foregoing methods is 50%.

In an embodiment, a method of treating patients with a tumor is provided, the method comprising: (a) administering to the patient an EGFR-directed therapeutic agent if the tumor is either AREG HIGH or EREG HIGH, wherein the tumor is considered AREG HIGH if the tumor has been histochemically or cytochemically determined to have a percentage of AREG+ tumor cells that is greater than or equal to a first pre-determined positive cut off and wherein the tumor is considered EREG HIGH if the tumor has been histochemically or cytochemically determined to have a percentage of EREG+ tumor cells that is greater than or equal to a second pre-determined positive cut off; and (b) administering to the patient a therapy course that does not include an EGFR-directed therapeutic agent if the tumor is both AREG LOW and EREG LOW, wherein the tumor is considered AREG LOW if the tumor has been histochemically or cytochemically determined to have a percentage of AREG+ tumor cells that is less than a first pre-determined negative cut off and wherein the tumor is considered EREG LOW if the tumor has been histochemically or cytochemically determined to have a percentage of EREG+ tumor cells that is less than a second pre-determined negative cut off.

In an embodiment, a method of selecting a treatment for a patient with a tumor is provided, the method comprising: (a) histochemically or cytochemically staining a sample of the tumor for human AREG protein; (b) histochemically or cytochemically staining a sample of the tumor for human EREG protein; (c) quantitating a percentage of AREG+ tumor cells in the sample of the tumor and comparing the percentage to a first pre-determined positive cut off and a first pre-determined negative cut off; (d) quantitating a percentage of EREG+ tumor cells in the sample of the tumor and comparing the percentage to a second pre-determined positive cut off and a second pre-determined negative cut off, (e) selecting the patient to receive a treatment course comprising an EGFR-directed therapeutic agent if the tumor is either AREG HIGH or EREG HIGH, wherein the tumor is considered AREG HIGH if the percentage of AREG+ tumor cells that is greater than or equal to the first pre-determined positive cut off and wherein the tumor is considered EREG HIGH if the percentage of EREG+ tumor cells is greater than or equal to the second pre-determined positive cut off; and (f) selecting the patient to receive a treatment course that does not include an EGFR-directed therapeutic agent if the tumor is both AREG LOW and EREG LOW, wherein the tumor is considered AREG LOW if the percentage of AREG+ tumor cells that is less than the first pre-determined negative cut off and wherein the tumor is considered EREG LOW if the percentage of EREG+ tumor cells is less than the second pre-determined positive cut off.

In some embodiments, the first pre-determined positive cut off is in the range of 30% to 50%, such as 30%, about 33.3%, 40%, and 50% and the first pre-determined negative cut off is in the range of 20% to 30%, such as 20%, 25%, and 30%.

In one specific embodiment, the first pre-determined positive cut off is 50% and the first pre-determined negative cut off is 20%.

In some embodiments, the second pre-determined positive cut off is in the range of 30% to 50%, such as 30%, about 33.3%, 40%, and 50% and the second pre-determined negative cut off is in the range of 20% to 30%, such as 20%, 25%, and 30%.

In one specific embodiment, the second pre-determined positive cut off is 50% and the second pre-determined negative cut off is 20%.

In one specific embodiment, the first and the second pre-determined positive cut offs are 50% and the first and second pre-determined negative cut offs are 20%.

In some embodiments, the tumor of the foregoing methods is a colorectal tumor.

In some embodiments, the EGFR-directed therapeutic agent of the foregoing methods is an anti-EGFR monoclonal antibody, such as cetuximab and/or panitumumab.

In some embodiments, the therapy of the foregoing methods further comprises administering to the patient a chemotherapy, such as a chemotherapy comprising irinotecan.

In some embodiments, the therapy of the foregoing methods that does not include the EGFR-directed therapeutic agent comprises administering to the patient a chemotherapy, such as a chemotherapy comprising irinotecan.

In some embodiments, the tumor of the foregoing methods does not comprise a detectable amount of a RAS protein with mutations that confer resistance to EGFR monoclonal antibody therapy.

In some embodiments, the tumor of the foregoing methods is RAS wild type (RAS-wt).

In some embodiments, the colorectal tumor of the foregoing methods is a left-sided tumor

In some embodiments, the colorectal tumor of the foregoing methods is a right-sided tumor.

In some embodiments of the foregoing methods, the sample is derived from a resection of a colorectal tumor.

In some embodiments of the foregoing methods, the sample is a biopsy sample of a colorectal tumor.

In some embodiments, the samples of the tumor of the foregoing methods are formalin-fixed paraffin-embedded tissue sections.

In some embodiments of the foregoing methods, the percentage of cells expressing EREG and the percentage of cells expressing AREG are quantitated by an automated method.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 provides a flow diagram demonstrating breakdown of study sample.

FIGS. 2A-2D illustrate the agreement between Algorithm and Consensus Pathologists' Scores for 30 PICCOLO Verification FOVs. FIG. 2A shows AREG Total Tumor Counts; FIG. 2B shows the AREG Percentage Positive; FIG. 2C shows the EREG Total Tumor Counts; and FIG. 2D shows the EREG Percentage Positive.

FIG. 3 illustrates a scatterplot of AREG versus EREG IHC percentage positivity. Four quadrants based on the 50% cut points are labeled: (A) AREG LOW/EREG HIGH; (B) AREG HIGH/EREG HIGH; (C) AREG LOW/EREG LOW; and (D) AREG HIGH/EREG LOW. High ligand expressors (Solid Dots; Quadrants A, B, and D) are defined as AREG and/or EREG percentage positivity above 50%; low ligand expressors (Open Dots; Quadrant C) are defined as AREG and EREG percentage positivity both below 50%. The 50% cut points are shown by dotted lines.

FIGS. 4A and 4B provide PFS Kaplan-Meier curves for RAS-wt patients. FIG. 4A shows the PFS Kaplan-Meier curves for RAS-wt patients in the low ligand expressor groups. FIG. 4B shows the PFS Kaplan-Meier curves for the RAS-wt patients in the high ligand expressor groups.

### DETAILED DESCRIPTION OF THE DISCLOSURE

This disclosure relates generally to methods, systems, and compositions for the histochemical staining and evaluation of colorectal tumor samples for EGFR and EGFR ligand expression. The disclosed methods, systems, and compositions are useful for, among other things, stratifying colorectal cancer patients according to a likelihood that their tumor will respond to an EGFR-directed therapeutic agent.

### I. Terms

Unless otherwise explained, all technical and scientific terms used herein have the same meaning as commonly understood by one of ordinary skill in the art to which a disclosed disclosure belongs. The singular terms “a,” “an,” and “the” include plural referents unless context clearly indicates otherwise.

Suitable methods and materials for the practice and/or testing of embodiments of the disclosure are described below. Such methods and materials are illustrative only and are not intended to be limiting. Other methods and materials similar or equivalent to those described herein can be used.

All publications, patent applications, patents, and other references mentioned herein are incorporated by reference in their entirety for all purposes. In case of conflict, the present specification, including explanations of terms, will control.

In order to facilitate review of the various embodiments of the disclosure, the following explanations of specific terms are provided:

Administer: To provide or give a subject an agent, for example, a composition, drug, etc., by any effective route. Exemplary routes of administration include, but are not limited to, oral, injection (such as subcutaneous, intramuscular, intradermal, intraperitoneal, and intravenous), sublingual, rectal, transdermal (e.g., topical), intranasal, vaginal and inhalation routes.

Antibody: A peptide (e.g., polypeptide) that includes at least a light chain or heavy chain immunoglobulin variable region and specifically binds an epitope of an antigen. Unless otherwise dictated by context, the term “antibody” shall be construed to explicitly include antibody fragments.

Antibody fragment: A molecule other than an intact antibody that comprises a portion of an intact antibody that binds the antigen to which the intact antibody binds. Examples of antibody fragments include but are not limited to Fv, Fab, Fab′, Fab′-SH, F(ab′)2; diabodies; linear antibodies; single-chain antibody molecules (e.g. scFv); and multispecific antibodies formed from antibody fragments.

Biomarker: As used herein, the term “biomarker” shall refer to any molecule or group of molecules found in a sample that can be used to characterize the sample or a subject from which the sample is obtained. For example, a biomarker may be a molecule or group of molecules whose presence, absence, or relative abundance is: characteristic of a particular disease state; indicative of the severity of a disease or the likelihood or disease progression or regression; and/or predictive that a pathological condition will respond to a particular treatment.

Biomarker-specific reagent: A specific binding agent that is capable of specifically binding directly to one or more biomarkers in the cellular sample or tissue sample. The phrase “[TARGET] biomarker-specific reagent” shall refer to a biomarker-specific reagent that is capable of specifically binding to the recited target biomarker.

Counterstaining: The staining of tissue sections with dyes that allow one to see the entire “landscape” of the tissue section and serve as a reference for the main color used for the detection of tissue targets. Such dyes can stain cell nuclei, the cell membrane, or the entire cell. Examples of dyes include DAPI, which binds to nuclear DNA and emits strong blue light; Hoechst blue stain, which binds to nuclear DNA and emits strong blue light; and Propidium iodide, which binds to nuclear DNA and emits strong red light. Counterstaining of the intracellular cytoskeletal network can be done using phalloidin conjugated to fluorescent dyes. Phalloidin is a toxin that tightly binds to actin filaments in a cell's cytoplasm, which then become clearly visible under the microscope.

Detectable moiety: A molecule or material that can produce a detectable signal (such as a visual, electrical, or other signal) that indicates the presence and/or concentration of the detectable moiety or label deposited on the sample. The detectable signal can be generated by any known or yet to be discovered mechanism including absorption, emission and/or scattering of a photon (including radio frequency, microwave frequency, infrared frequency, visible frequency and ultra-violet frequency photons). Exemplary detectable moieties include (but are not limited to) chromogenic, fluorescent, phosphorescent, and luminescent molecules and materials, catalysts (such as enzymes) that convert one substance into another substance to provide a detectable difference (such as by converting a colorless substance into a colored substance or vice versa, or by producing a precipitate or increasing sample turbidity). In some examples, the detectable moiety is a fluorophore, which belongs to several common chemical classes including coumarins, fluoresceins (or fluorescein derivatives and analogs), rhodamines, resorufins, luminophores and cyanines. Additional examples of fluorescent molecules can be found in Molecular Probes Handbook—A Guide to Fluorescent Probes and Labeling Technologies, Molecular Probes, Eugene, Oreg., ThermoFisher Scientific, 11th Edition. In other embodiments, the detectable moiety is a molecule detectable via brightfield microscopy, such as dyes including diaminobenzidine (DAB), 4-(dimethylamino) azobenzene-4′-sulfonamide (DABSYL), tetramethylrhodamine (DISCOVERY Purple), N,N′-biscarboxypentyl-5,5′-disulfonato-indo-dicarbocyanine (Cy5), and Rhodamine 110 (Rhodamine).

Detection reagent: Any reagent used to deposit a detectable moiety in proximity to a biomarker-specific reagent bound to a biomarker in a cellular sample to thereby stain the sample. Non-limiting examples include secondary detection reagents (such as secondary antibodies capable of binding to a primary antibody, anything that specifically binds biotin or avidin), tertiary detection reagents (such as tertiary antibodies capable of binding to secondary antibodies), enzymes directly or indirectly associated with the specific binding agent, chemicals reactive with such enzymes to effect deposition of a fluorescent or chromogenic stain, wash reagents used between staining steps, and the like.

Monoclonal antibody: An antibody preparation having a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to a polyclonal antibody, each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method.

Polyclonal antibody: An antibody preparation that typically includes different antibodies directed against different determinants (epitopes).

Sample: Any material obtained for a diagnostic purpose from a subject and processed in a manner compatible with testing for the presence or absence and/or the amount of a biomarker in the material using a specific binding agent. Examples of diagnostic purposes include: diagnosing or prognosing disease in the subject, and/or predicting response of a disease to a particular therapeutic regimen, and/or monitoring a subject's response to a therapeutic regimen, and/or monitoring for progression or recurrence of disease.
(a) Cellular sample: A sample containing intact cells, such as cell cultures, blood or other body fluid samples containing cells, cell smears (such as Pap smears and cervical monolayers), fine needle aspirates (FNA), liquid based cytology samples, and surgical specimens taken for pathological, histological, or cytological interpretation.
(b) Tissue sample: A cellular sample that preserves the cross-sectional spatial relationship between the cells as they existed within the subject from which the sample was obtained. “Tissue sample” shall encompass both primary tissue samples (i.e. cells and tissues produced by the subject) and xenografts (i.e. foreign cellular samples implanted into a subject).

Section: When used as a noun, a thin slice of a tissue sample suitable for microscopic analysis, typically cut using a microtome. When used as a verb, making a section of a tissue sample, typically using a microtome.

Serial Section: Any one of a series of sections cut in sequence from a tissue sample. For two sections to be considered “serial sections” of one another, they do not necessarily need to be consecutive sections from the tissue, but they should generally contain the same tissue structures in the same cross-sectional relationship, such that the structures can be matched to one another after histological staining.

Specific Binding: As used herein, the phrase “specific binding,” “specifically binds to,” or “specific for” refers to measurable and reproducible interactions such as binding between a target and a specific binding agent, which is determinative of the presence of the target in the presence of a heterogeneous population of molecules including biological molecules. For example, a binding entity that specifically binds to a target may be an antibody that binds the target with greater affinity, avidity, more readily, and/or with greater duration than it binds to other targets.

Specific binding agent: Any composition of matter that is capable of specifically binding to a target chemical structure associated with a cellular sample or tissue sample (such as a biomarker expressed by the sample or a biomarker-specific reagent bound to the sample). Examples include but are not limited to nucleic acid probes specific for particular nucleotide sequences; antibodies and antigen binding fragments thereof; and engineered specific binding structures, including ADNECTINs (scaffold based on 10th FN3 fibronectin; Bristol-Myers-Squibb Co.), AFFIBODYs (scaffold based on Z domain of protein A from S. aureus; Affibody AB, Solna, Sweden), AVIMERs (scaffold based on domain A/LDL receptor; Amgen, Thousand Oaks, Calif.), dAbs (scaffold based on VH or VL antibody domain; GlaxoSmithKline PLC, Cambridge, UK), DARPins (scaffold based on Ankyrin repeat proteins; Molecular Partners AG, Zurich, CH), ANTICALINs (scaffold based on lipocalins; Pieris AG, Freising, DE), NANOBODYs (scaffold based on VHH (camelid Ig); Ablynx NN, Ghent, BE), TRANS-BODYs (scaffold based on Transferrin; Pfizer Inc., New York, N.Y.), SMIPs (Emergent Biosolutions, Inc., Rockville, Md.), and TETRANECTINs (scaffold based on C-type lectin domain (CTLD), tetranectin; Borean Pharma A/S, Aarhus, DK). Descriptions of such engineered specific binding structures are reviewed by Wurch et al., Development of Novel Protein Scaffolds as Alternatives to Whole Antibodies for Imaging and Therapy: Status on DISCOVERY Research and Clinical Validation, Current Pharmaceutical Biotechnology, Vol. 9, pp. 502-509 (2008), the content of which is incorporated by reference.

Stain: When used as a noun, the term “stain” shall refer to any substance that can be used to visualize specific molecules or structures in a cellular sample for microscopic analysis, including brightfield microscopy, fluorescent microscopy, electron microscopy, and the like. When used as a verb, the term “stain” shall refer to any process that results in deposition of a stain on a cellular sample.

Subject: A mammal from which a sample has been obtained or derived. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the subject is a human.

### II. Histochemically or Cytochemically Labeling Samples for EGFR Ligands

The present methods are based on histochemically or cytochemically staining tumor samples for human EREG protein and human AREG protein.

A. Samples and Sample Preparation

The present methods are performed on tissue samples or cytological preparations of tissue samples obtained from a tumor, including, for example, tumor biopsies samples, resection samples, cell smears, fine needle aspirates (FNA), liquid based cytology samples, and the like.

In an embodiment, the sample is a fixed tissue sample. Fixing a tissue sample preserves cells and tissue constituents in as close to a life-like state as possible and allows them to undergo preparative procedures without significant change. Autolysis and bacterial decomposition processes that begin upon cell death are arrested, and the cellular and tissue constituents of the sample are stabilized so that they withstand the subsequent stages of tissue processing. Fixatives can be classified as cross-linking agents (such as aldehydes, e.g., formaldehyde, paraformaldehyde, and glutaraldehyde, as well as non-aldehyde cross-linking agents), oxidizing agents (e.g., metallic ions and complexes, such as osmium tetroxide and chromic acid), protein-denaturing agents (e.g., acetic acid, methanol, and ethanol), fixatives of unknown mechanism (e.g., mercuric chloride, acetone, and picric acid), combination reagents (e.g., Carnoy's fixative, methacarn, Bouin's fluid, B5 fixative, Rossman's fluid, and Gendre's fluid), microwaves, and miscellaneous fixatives (e.g., excluded volume fixation and vapor fixation). Additives may also be included in the fixative, such as buffers, detergents, tannic acid, phenol, metal salts (such as zinc chloride, zinc sulfate, and lithium salts), and lanthanum. The most commonly used fixative in preparing samples is formaldehyde, generally in the form of a formalin solution (formaldehyde in an aqueous (and typically buffered) solution). In an embodiment, the samples used in the present methods are fixed by a method comprising fixation in a formalin-based fixative. In one example, the fixative is 10% neutral buffered formalin. Notwithstanding these examples, the tissues can be fixed by process using any fixation medium that is compatible with the biomarker-specific reagents and specific detection reagents used.

In some examples, the fixed tissue sample is embedded in an embedding medium. An embedding medium is an inert material in which tissues and/or cells are embedded to help preserve them for future analysis. Embedding also enables tissue samples to be sliced into thin sections. Embedding media include paraffin, celloidin, OCT™ compound, agar, plastics, or acrylics. In an embodiment, the sample is fixed in formalin and embedded in paraffin to form a formalin-fixed, paraffin-embedded (FFPE) block. In a typical embedding process (such as used for FFPE blocks), after the sample is fixed it is subjected to a series of alcohol immersions, typically using increasing alcohol concentrations ranging from about 70% to about 100%, to dehydrate the sample. The alcohol generally is an alkanol, particularly methanol and/or ethanol. Particular working embodiments have used 70%, 95% and 100% ethanol for these serial dehydration steps. After the last alcohol treatment step the sample is then immersed into another organic solvent, commonly referred to as a clearing solution. The clearing solution (1) removes residual alcohol, and (2) renders the sample more hydrophobic for a subsequent waxing step. The clearing solvent typically is an aromatic organic solvent, such as xylene. Blocks are formed by applying the embedding material to the cleared sample, from which tissue sections can be cut (such as by using a microtome).

Notwithstanding these examples, no specific processing step is required by the present disclosure, so long as the tissue sample or cytology sample obtained is compatible with staining of the sample for the biomarkers of interest and the reagents used for that staining and subsequent microscopic evaluation or digital imaging to quantitate the number of cells expressing EREG or AREG.

B. Sample Selection

B1. Tumor Stage

In an embodiment, the tumor from which the sample is derived is staged prior to being stained for the EREG and/or AREG protein(s). Stage 0 colorectal cancers are cancers that have not grown beyond the inner lining of the colon. Stage I colorectal cancers are cancers that have not spread outside of the colon wall itself or into nearby lymph nodes. Stage II colorectal cancers are cancers that have grown through the wall of the colon, and possibly into nearby tissue, but have not yet spread to the lymph nodes. Stage III colorectal cancers are cancers that have spread to nearby lymph nodes, but have not yet spread to other parts of the body. Stage IV colorectal cancers are cancers that have spread from the colon to distant organs and tissues. In an embodiment, the sample is selected for staining if it is a stage III or a stage IV colorectal cancer. In another embodiment, the sample is selected for staining if it is a stage IV colorectal cancer.

B2. EGFR-Resistance Mutations

The tumor may be screened for the presence of mutations that confer resistance to EGFR-directed therapeutic agents. Examples of such mutations include activating mutations in the RAS oncogene (see, e.g., Prior, Waring, Vale, Kaprapetis, Douillard, and Van Cutsem) and BRAF gene (see, e.g., Bokemeyer). In some embodiments, the sample or subject has been determined to be RAS wild type before staining for EGFR ligands is performed. As used herein, a “wild-type RAS” shall mean that the sample or subject has tested negative in a RAS mutation screening assay for mutations within at least NRAS and KRAS that confer resistance to EGFR monoclonal antibody therapy (whether currently known or later discovered). In an embodiment, the RAS mutation screening assay comprises determining the presence or absence of activating mutations in at least codons 12 and 13 of NRAS and codons 12 and 13 of KRAS, wherein the sample is considered “RAS wild type” if the samples or subject is free of activating mutations of each of codons 12 and 13 of NRAS and codons 12 and 13 of KRAS. In another embodiment, the RAS mutation screening assay comprises determining the presence or absence of activating mutations in at least codons 12, 13, 59, 61, 117, and 146 of NRAS and codons 12, 13, 59, 61, 117, and 146 of KRAS, wherein the sample is considered “RAS wild type” if the samples or subject is free of activating mutations of each of codons 12, 13, 59, 61, 117, and 146 of NRAS and codons 12, 13, 59, 61, 117, and 146 of KRAS are determined to have wild-type RAS status. Screening for Ras mutation status may be performed on a variety of different types of samples from the subject, including tissue samples derived from the tumor and blood samples from the same subject from which the tissue sample has been obtained. Many different methods for screening for Ras mutational status are known, including methods based on sequencing, pyrosequencing, real-time PCR, allele-specific real-time PCR, Restriction fragment length polymorphism (RFLP) analysis with sequencing, amplification refractory mutation systems (ARMS), or COLD-PCR (coamplification at lower denaturation temperature PCR) with sequencing. Other specific exemplary methods of screening for Ras mutations include, but are not limited to: blood-based screening methods relying on circulating tumor DNA (ctDNA) (see, for example, Schmiegel et al., Mol. Oncol., Vol. 11, Issue 2, pp. 208-19 (February 2017) (screening for mutations by applying an emulsion digital PCR-based assay for exons 2, 3, and 4 of KRAS and NRAS to circulating cell-free DNA assay)) and tissue-based methods, such as screening for mutations in KRAS and NRAS exons 2, 3, and 4 in tumor tissue samples using Sanger sequencing, massively parallel sequencing (including sequencing methodologies based on pyrosequencing, cyclic reversible termination, semiconductor sequencing, or phospholinked fluorescent nucleotide technologies), or PCR-based assays (including quantitative PCR and digital PCR). The present disclosure is not limited to any particular method for screening for Ras mutation status.

B3. Sidedness

In some embodiments, the sidedness of the colorectal tumor is determined prior to staining. Colorectal tumors can be divided into right sided tumors (tumors occurring from the caecum to the splenic flexure) and left-sided tumors (tumors occurring from the splenic flexure to the rectum). As shown in the Examples, the present scoring methods are useful in both left-sided and right-sided tumors. It should be noted that right-sidedness of the tumor is typically a negative predictor for response to EGFR-directed therapeutic agents (see, e.g. Tejpar). As shown in the present examples, however, the present scoring methods are useful in predicting response in both left- and right-sided tumors.

B4. EGFR Status

In some embodiments, the EGFR status of the tumor is determined. Any method of determining EGFR status may be used (whether currently known or developed in the future). In a specific example, the EGFR status is determined by immunohistochemistry (IHC) or immunocytochemistry (ICC). Canonical amino acid sequence for full length human EGFR is set forth at SEQ ID NO: 1. As would be understood by a person of ordinary skill in the art, the precise amino acid sequences may vary from subject-to-subject. In an embodiment, the IHC or ICC assay is perform with an antibody capable of specifically binding to a polypeptide comprising SEQ ID NO: 1. Non-limiting examples of an EGFR-specific monoclonal antibodies are set forth in Table 1

TABLE 1

| Clone | Species | Epitope/Immunogen | Reference | Manufacturer |
|---|---|---|---|---|
| 5B7 | Rabbit | Intracellular | Mascaux et | Ventana |
| | | epitope located | al., Clin. | Medical |
| | | in the suppressor | Cancer Res., | Systems, |
| | | of cytokine | vol. 17, | Inc. |
| | | signaling 3 | issue 24, | |
| | | (SOCS3) protein | pp. 7796-7807 | |
| | | binding site | (Dec. 2011) | |
| EGFR.25 | Mouse | 200 amino | Shinojima et al., | Leica |
| | | acids of the | Cancer Res., | Biosystems |
| | | intracellular | Vol. 63, | Newcastle |
| | | domain of the | Issue 20 | Ltd. |
| | | EGFR molecule, | (Oct. 2003). | |
| | | excluding the | | |
| | | conserved tyrosine | | |
| | | kinase domain | | |
| 2-18C9 | Mouse | Extracellular | Spaulding & | Dako |
| | | epitope located | Spaulding, | |
| | | near ligand | Seminars | |
| | | binding site and | in Oncology, | |
| | | conserved in | Vol. 29, | |
| | | EGFRVIII | Issue 5, | |
| | | truncated mutants | Supplement 14, | |
| | | | pp. 45-54 (Oct. | |
| | | | 2002) | |
| EGFR.113 | Mouse | Immunogen | Shinojima et al., | Leica |
| | | comprising | Cancer Res., | Biosystems |
| | | extracellular | Vol. 63, | Newcastle |
| | | domain | Issue 20 (Oct. | Ltd. |
| | | | 2003). | |
| H11 | Mouse | Immunogen is | Anagnostou | Thermo- |
| | | HC2 20 d2 | et al., | Fisher |
| | | cells; recognizes | Cancer | Biocare |
| | | epitope located | epidemiology, | |
| | | in extracellular | biomarkers & | |
| | | domain and | prevention, | |
| | | conserved in | Vol. 19, | |
| | | EGFRvIII | Issue 4, pp. | |
| | | truncated mutants | 982-991 | |
| | | | (2010) | |
| 15F8 | Rabbit | Produced by | Anagnostou | Cell |
| | | immunizing | et al., | Signaling |
| | | animals with | Cancer | Technology, |
| | | a synthetic | epidemiology, | Inc. |
| | | peptide | biomarkers & | |
| | | corresponding to | prevention, | |
| | | residues near | Vol. 19, | |
| | | the carboxy | Issue 4, | |
| | | terminus of | pp. 982-991 | |
| | | human EGF | (2010) | |
| | | receptor. | Cell Signaling | |
| | | | Product Data | |
| | | | Sheet | |

In an embodiment, the EGFR biomarker-specific reagent is a monoclonal antibody directed against an intracellular domain of EGFR. In another embodiment, the EGFR biomarker-specific reagent is a monoclonal antibody directed against an extracellular domain of EGFR. In another embodiment, the EGFR biomarker-specific reagent is a monoclonal antibody that recognizes both full length EGFR and EGFRvIII mutant.

C. AREG and EREG Histochemical and cytochemical Staining

Labeling of EREG and AREG may be accomplished by contacting a tissue section or cytological preparation with a biomarker-specific reagent under conditions that facilitate specific binding between the target biomarker and the biomarker-specific reagent. The sample is then contacted with a set of detection reagents that interact with the biomarker-specific reagent to facilitate deposition a detectable moiety in close proximity the target biomarker on the sample, thereby generating a detectable signal localized to the target biomarker. Biomarker-stained samples may optionally be additionally stained with a contrast agent (such as a hematoxylin stain) to visualize macromolecular structures.

The histochemical and cytochemical staining methods disclosed herein comprise contacting a tissue section or cytological preparation of a colorectal tumor with one or more biomarker-specific reagents for human EREG protein and human AREG protein under conditions that support specific binding between biomarker-specific reagents and the biomarkers expressed by the sample. EREG and AREG are expressed first as a pro-peptide, which is cleaved at the cell surface to release an active signaling domain. Canonical amino acid sequences for human EREG and AREG (and pro-peptides thereof) are set forth in Table 2. As would be understood by a person of ordinary skill in the art, the precise amino acid sequences may vary from subject-to-subject.

TABLE 2

| BIOMARKER | UNIPROT ID | SEQ ID NO |
|---|---|---|
| Proepiregulin | O14944-1 | 2 |
| Mature Epiregulin | O14944-1 | 2 (aa 63-108) |
| Amphiregulin Pro-peptide | P15514-1 | 3 |
| Mature Amphiregulin | P15514-1 | 3 (aa 101-187) |

In an embodiment, the biomarker-specific reagent to human EREG protein is a biomarker-specific reagent capable of specifically binding to a polypeptide comprising SEQ ID NO: 2. In an embodiment, the biomarker-specific reagent to human AREG protein is a biomarker-specific reagent capable of specifically binding to a polypeptide comprising SEQ ID NO: 3.

In an embodiment, the EREG biomarker-specific reagent is an antibody. Non-limiting examples of an EREG-specific antibodies are set forth in Table 3:

TABLE 3

| | | Epitope/ | | |
|---|---|---|---|---|
| Clone | Species | Immunogen | Reference | Manufacturer |
| J5H1L1 | Rabbit | Immunogen | WO 2017-001350 | Not |
| | Mono- | comprising | A1 (incorporated | commercially |
| | clonal | amino acid | by reference) | available |
| | | residues 148- | | |
| | | 169 of SEQ | | |
| | | ID NO: 2. | | |
| | | Does not | | |
| | | recognize mature | | |
| | | EREG. | | |
| J89H12L3 | Rabbit | Immunogen | WO 2017-001350 | Not |
| | Mono- | comprising | A1 (incorporated | commercially |
| | clonal | amino acid | by | available |
| | | residues 156- | reference) | |
| | | 169 of SEQ | | |
| | | ID NO: 2. | | |
| | | Does not | | |
| | | recognize mature | | |
| | | EREG. | | |
| J89H12L8 | Rabbit | Immunogen | WO 2017-001350 | Not |
| | Mono- | comprising | A1 (incorporated | commercially |
| | clonal | amino acid | by | available |
| | | residues 156- | reference) | |
| | | 169 of SEQ | | |
| | | ID NO: 2. | | |
| | | Does not | | |
| | | recognize mature | | |
| | | EREG. | | |
| D4O5I | Rabbit | Raised against | CST product | Cell |
| | Mono- | residues | insert | Signaling |
| | clonal | surrounding | | Technology, |
| | | Glu155. | | Inc. |
| | | Recognizes | | |
| | | endogenous levels | | |
| | | of proepiregulin | | |
| | | and the | | |
| | | C-terminal | | |
| | | propeptide of the | | |
| | | EREG protein. It | | |
| | | does not recognize | | |
| | | the mature | | |
| | | form of EREG. | | |

In an embodiment, the EREG biomarker-specific reagent is a monoclonal antibody selected from Table 3.

In an embodiment, the AREG biomarker-specific reagent is an antibody. Non-limiting examples of an AREG-specific antibodies are set forth in Table 4:

TABLE 4

| | | Epitope/ | | |
|---|---|---|---|---|
| Clone | Species | Immunogen | Reference | Manufacturer |
| J111H1L10 | Rabbit | Immunogen | WO | Not |
| | Mono- | comprising | 2017-001350 | commercially |
| | clonal | amino acid | A1 | available |
| | | residues 238- | (incorporated | |
| | | 252 of SEQ | by reference) | |
| | | ID NO: 3. | | |
| | | Does not | | |
| | | recognize | | |
| | | mature | | |
| | | AREG. | | |
| G-4 | Mouse | Raised against | Santa Cruz | Santa Cruz |
| | mono- | amino acids | Datasheet | Biotechnology, |
| | clonal | 1-155 of | | Inc. |
| | | amphiregulin of | | |
| | | human origin | | |
| AF262 | Goat | Val107-Lys184 | R&D | R&D Systems |
| | poly- | of UNIPROT | Systems | |
| | clonal | Accession No. | Datasheet | |
| | | P15514 | | |

In an embodiment, the AREG biomarker-specific reagent is selected from Table 4.

After binding to the sample, the biomarker-specific reagents are visualized using a set of detection reagents. In some embodiments, the detection reagents deposit a stain that is compatible with brightfield microscopy. Non-limiting examples of commercially available detection reagents or kits comprising detection reagents useful in the present methods include: VENTANA ultraView detection systems (secondary antibodies conjugated to enzymes, including HRP and AP); VENTANA iVIEW detection systems (biotinylated anti-species secondary antibodies and streptavidin-conjugated enzymes); OptiView detection systems (anti-species secondary antibody conjugated to a hapten and an anti-hapten tertiary antibody conjugated to an enzyme multimer); VENTANA Amplification kit (unconjugated secondary antibodies, which can be used with any of the foregoing VENTANA detection systems to amplify the number of enzymes deposited at the site of primary antibody binding); VENTANA OptiView Amplification system (Anti-species secondary antibody conjugated to a hapten, an anti-hapten tertiary antibody conjugated to an enzyme multimer, and a tyramide conjugated to the same hapten); VENTANA DISCOVERY (e.g. DISCOVERY Yellow Kit, DISCOVERY Purple Kit, DISCOVERY Silver kit, DISCOVERY Red Kit, DISCOVERY Rhodamine Kit, etc.) DISCOVERY OmniMap, DISCOVERY UltraMap anti-hapten antibody, secondary antibody, chromogen, fluorophore, and dye kits, each of which are available from Ventana Medical Systems, Inc. (Tucson, Ariz.); PowerVision and PowerVision+IHC Detection Systems (secondary antibodies directly polymerized with HRP or AP into compact polymers bearing a high ratio of enzymes to antibodies); and DAKO EnVision™+ System (enzyme labeled polymer that is conjugated to secondary antibodies).

The histochemical or cytochemical methods herein may be performed on an automated staining machine (or other slide processing machine), manually, or feature a combination of automated steps and manual steps. In an embodiment, the histochemical and cytochemical staining methods described herein are performed on an automated IHC staining device. Specific examples of automated IHC staining devices include: intelliPATH (Biocare Medical), WAVE (Celerus Diagnostics), DAKO OMNIS and DAKO AUTOSTAINER LINK 48 (Agilent Technologies), BENCHMARK XT (Ventana Medical Systems, Inc.), BENCHMARK Special Stains (Ventana Medical Systems, Inc.), BENCHMARK ULTRA (Ventana Medical Systems, Inc.), BENCHMARK GX (Ventana Medical Systems, Inc.), DISCOVERY XT (Ventana Medical Systems, Inc.), DISCOVERY ULTRA (Ventana Medical Systems, Inc.), Leica BOND, and Lab Vision Autostainer (Thermo Scientific). Automated IHC staining device are also described by Prichard, Overview of Automated Immunohistochemistry, Arch Pathol Lab Med., Vol. 138, pp. 1578-1582 (2014), incorporated herein by reference in its entirety. Additionally, Ventana Medical Systems, Inc. is the assignee of a number of United States patents disclosing systems and methods for performing automated analyses, including U.S. Pat. Nos. 5,650,327, 5,654,200, 6,296,809, 6,352,861, 6,827,901 and 6,943,029, and U.S. Published Patent Application Nos. 20030211630 and 20040052685, each of which is incorporated herein by reference in its entirety. The methods of the present disclosure may be adapted to be performed on any appropriate automated IHC staining device.

The present disclosure is not limited to the use of automated systems. In some embodiments, the histochemical labeling methods described herein are applied manually. Or, particular steps may be performed manually while other steps are performed in an automated system.

If desired, the biomarker-stained slides may be counterstained to assist in identifying morphologically relevant areas and/or for identifying regions of interest (ROIs). Examples of counterstains include chromogenic nuclear counterstains, such as hematoxylin (stains from blue to violet), Methylene blue (stains blue), toluidine blue (stains nuclei deep blue and polysaccharides pink to red), nuclear fast red (also called Kernechtrot dye, stains red), and methyl green (stains green); non-nuclear chromogenic stains, such as eosin (stains pink); fluorescent nuclear stains, including 4′, 6-diamino-2-pheylindole (DAPI, stains blue), propidium iodide (stains red), Hoechst stain (stains blue), nuclear green DCS1 (stains green), nuclear yellow (Hoechst 5769121, stains yellow under neutral pH and stains blue under acidic pH), DRAQ5 (stains red), DRAQ7 (stains red); fluorescent non-nuclear stains, such as fluorophore-labeled phalloidin, (stains filamentous actin, color depends on conjugated fluorophore).

In certain embodiments, a serial section of the biomarker-stained section (or the biomarker-stained section itself) may be morphologically stained. Basic morphological staining techniques often rely on staining nuclear structures with a first dye, and staining cytoplasmic structures with a second stain. Many morphological stains are known, including but not limited to, hematoxylin and eosin (H&E) stain and Lee's Stain (Methylene Blue and Basic Fuchsin). Examples of commercially available H&E stainers include the VENTANA SYMPHONY (individual slide stainer) and VENTANA HE 600 (individual slide stainer) H&E stainers from Roche; the Dako CoverStainer (batch stainer) from Agilent Technologies; the Leica ST4020 Small Linear Stainer (batch stainer), Leica ST5020 Multistainer (batch stainer), and the Leica ST5010 Autostainer XL series (batch stainer) H&E stainers from Leica Biosystems Nussloch GmbH.

### III. Cell Quantitation

The present scoring algorithms are based on quantitating a percentage of both EREG-stained cells (EREG+ tumor cells) and AREG-stained cells (AREG+ tumor cells). The percentage of positively stained cells may be determined manually or by automated methods.

In a manual method, a skilled user (such as a pathologist) observes a magnified image of the stained sample and estimates the percentage of cells within the field of view that stain positively for the respective marker. Where the sample is a tissue section, the skilled reader may identify a region of interest (ROI) within the field of view (FOV) for analysis (such as identifying the tumor margin and evaluating only cells within the tumor margin), or may simply evaluate all cells within the FOV.

In an embodiment, an automated quantification is performed on a digital pathology systems. There are two basic components of digital pathology systems: (1) a scanning system for generating digital images of a stained sample; and (2) an image analysis system for identifying and quantifying specific features within the image. In an exemplary digital pathology workflow of the present methods, the stained sample is digitized on a staining platform and then analyzed by the image analysis system to identify and quantify features within the image that correspond to the total number of cells and the total number of biomarker-stained cells. The percentage of cells staining positively for the respective markers may then be derived from these two values. This could take a number of different forms. For example, the system may identify objects within the image that correspond to “cells” and then identify how many of those cells contain the appropriate staining pattern indicative of positive biomarker staining. Alternatively, the system could calculate the area of the image and the percentage of that area that correlates with positive biomarker staining. In yet another embodiment, the system could identify all areas of the image that correspond to cell membranes and calculate the percentage of that area that corresponds to positive biomarker staining. Many other arrangements could be imagined or used. The output of both the manual and automated methods will be a percentage of EREG+ tumor cells and a percentage of AREG+ tumor cells.

A detailed overview of various scanners (both fluorescent and brightfield) for digital pathology applications can be found at Farahani et al., Whole slide imaging in pathology: advantages, limitations, and emerging perspectives, Pathology and Laboratory Medicine Intl, Vol. 7, p. 23-33 (June 2015), the content of which is incorporated by reference in its entirety. Examples of commercially available slide scanners include: 3DHistech PANNORAIVIIC SCAN II; DigiPath PATHSCOPE; Hamamatsu NANOZOOMER RS, HT, and XR; Huron TISSUESCOPE 4000, 4000XT, and HS; Leica SCANSCOPE AT, AT2, CS, FL, and SCN400; Mikroscan D2; Olympus VS120-SL; Omnyx VL4, and VL120; PerkinElmer LAMINA; Philips ULTRA-FAST SCANNER; Sakura Finetek VISIONTEK; Unic PRECICE 500, and PRECICE 600x; VENTANA ISCAN COREO and ISCAN HT; and Zeiss AXIO SCAN.Z1. Other exemplary systems and features can be found in, for example, WO2011-049608) or in U.S. Patent Application No. 61/533,114, filed on Sep. 9, 2011, entitled IMAGING SYSTEMS, CASSETTES, AND METHODS OF USING THE SAME the content of which is incorporated by reference in its entirety.

Exemplary commercially-available image analysis software packages useful for implementing the automated methods as disclosed herein include VENTANA VIRTUOSO software suite (Ventana Medical Systems, Inc.); TISSUE STUDIO, DEVELOPER XD, and IMAGE MINER software suites (Definiens); BIOTOPIX, ONCOTOPIX, and STEREOTOPIX software suites (Visiopharm); and the HALO platform (Indica Labs, Inc.).

### IV. Treatment Selection

Treatment regimens are selected by comparing the percentage of both EREG-stained cells (EREG+ tumor cells) and AREG-stained cells (AREG+ tumor cells) to pre-determined cut offs.

The pre-determined cut off used could be indicative of a likelihood that the patient will have a positive response to the EGFR-directed therapy or a cut off that is indicative of a likelihood of that the patient will have a negative response to the EGFR-directed therapy. As used herein, a “positive response to the EGFR-directed therapy” means that the cut off is associated with an improvement in at least one of overall survival and progression-free survival after treatment with the EGFR-directed therapy. As used herein, a “negative response to the EGFR-directed therapy” means that the cut off is associated with a worsening in at least one of overall survival and progression-free survival after treatment with the EGFR-directed therapy.

In an embodiment, a cut off associated with a positive response to the EGFR-directed therapeutic agent is used. Separate cut offs are developed for EREG and AREG, and the percentage of EREG+ tumor cells is compared to the EREG-specific cut off and the percentage of AREG+ tumor cells is compared to the AREG-specific cut off. The patient is selected to receive the EGFR-directed therapy if either the percentage of the EREG+ tumor cells exceeds the pre-determined cut off for EREG or the percentage of the AREG+ tumor cells exceeds the pre-determined cut off for AREG.

In an embodiment, a cut off associated with a negative response to the EGFR-directed therapeutic agent is used. Separate cut offs are developed for EREG and AREG, and the percentage of EREG+ tumor cells is compared to the EREG-specific cut off and the percentage of AREG+ tumor cells is compared to the AREG-specific cut off. The patient is selected to receive the EGFR-directed therapy if either the percentage of the EREG+ tumor cells exceeds the pre-determined cut off for EREG or the percentage of the AREG+ tumor cells exceeds the pre-determined cut off for AREG. A therapeutic course that does not include the EGFR-directed therapeutic agent is selected for the patient if both the percentage of the EREG+ tumor cells and the percentage of the AREG+ tumor cells fall below their respective pre-determined cut offs.

In another embodiment, two cut offs are used for each marker: (a) a cut off associated with a positive response to the EGFR-directed therapeutic agent (“positive cut off”); and (b) a cut off associated with a negative response to the EGFR-directed therapeutic agent (“negative cut off”). Separate cut offs are developed for EREG and AREG, the percentage of EREG+ tumor cells is compared to both the positive EREG-specific cut off and the negative EREG-specific cut off, and the percentage of AREG+ tumor cells is compared to both the positive AREG-specific cut off and the negative AREG-specific cut off. Treatment selection is as shown in Table 5:

TABLE 5

| | Scoring Result | Treatment selected |
|---|---|---|
| A | Either EREG+ or AREG+ | Administer the |
| | percentage exceeds the | EGFR-directed therapeutic |
| | respective positive cut off | agent |
| B | Both EREG+ and | Do not administer |
| | AREG+ percentage | the EGFR-directed |
| | is below the respective | therapeutic agent |
| | negative cut off | |
| C | Neither A nor B | Select therapeutic course |
| | | based on other factors |

Exemplary other factors include the availability and efficacy of immunotherapy for mismatch repair deficient tumors, angiogenesis inhibitors for patients with right-sided primaries, and BRAF and MEK inhibitors for BRAF-mutant tumors.

In an embodiment, the anti-EGFR therapeutic agent is an EGFR antibody. These therapies typically rely on antibodies or antibody fragments that bind to an extracellular domain of EGFR. In an embodiment, the EGFR antibody-based therapy comprises cetuximab and/or panitumumab.

In an embodiment, the EGFR-directed therapeutic agent is incorporated into a treatment regime for a RAS wild-type subject having a stage III colorectal tumor. Surgical removal of the tumor or a partial colectomy (including removal of nearby lymph nodes) followed by adjuvant chemotherapy and/or radiation therapy is typically performed at this stage, although chemotherapy (optionally in combination with radiation therapy) may be used without surgery for certain patients. Non-limiting combination therapies used at this stage include FOLFOX (5-FU, leucovorin, and oxaliplatin) or CapeOx (capecitabine and oxaliplatin). In one specific non-limiting embodiment, a method of treating a stage III colorectal cancer may comprise:
for patients selected to receive the EGFR-directed therapeutic agent: administering the EGFR antibody-based therapy, optionally in combination fluoropyrimidine-based chemotherapy or a fluoropyrimidine-based combination chemotherapy (such as FOLFOX or CapeOx); or
for patients selected not to receive the EGFR-directed therapeutic agent: administering a therapy course that does not comprise the EGFR antibody-based.

In another embodiment, the EGFR-directed therapeutic agent is incorporated into a treatment regime for a RAS wild-type subject having a stage IV colorectal tumor. Therapeutic regimes for stage IV colorectal tumors typically include surgical removal of the tumor or a partial colectomy (including removal of nearby lymph nodes) and metastases (if possible) and adjuvant or neoadjuvant chemotherapy and/or radiation therapy. Surgical removal of the tumor or a partial colectomy (including removal of nearby lymph nodes) and metastases (if possible), as well as chemotherapy and/or radiation therapy is typically performed at this stage. Common chemotherapies include fluoropyrimidine-based chemotherapies, optionally in combination with leucovorin and/or other chemotherapies and/or targeted therapies. Non-limiting combination therapies used at this stage include:
FOLFOX: leucovorin, 5-FU, and oxaliplatin (ELOXATIN);
FOLFIRI: leucovorin, 5-FU, and irinotecan (CAMPTOSAR);
CapeOX: capecitabine (XELODA) and oxaliplatin;
FOLFOXIRI: leucovorin, 5-FU, oxaliplatin, and irinotecan;
One of the above combinations plus either a drug that targets VEGF (such as bevacizumab [AVASTIN], ziv-aflibercept [ZALTRAP], or ramucirumab [CYRAMZA]), or a drug that targets EGFR (such as cetuximab [Erbitux] or panitumumab [VECTIBIX]);
5-FU and leucovorin, with or without a targeted drug;
Capecitabine, with or without a targeted drug;
Irinotecan, with or without a targeted drug;
Cetuximab alone;
Panitumumab alone;
Regorafenib (STIVARGA) alone; and
Trifluridine and tipiracil (LONSURF),
   In one specific non-limiting embodiment, a method of treating a stage IV colorectal cancer may comprise:
for subjects selected to receive the EGFR-directed therapeutic agent, administering the EGFR antibody-based therapy, optionally in combination with one or more additional therapies selected from the group consisting of FOLFOX, FOLFIRI, CapeOX, FOLFOXIRI, 5-FU and leucovorin, capecitabine, irinotecan, and a drug that targets VEGF (such as bevacizumab, ziv-aflibercept, and ramucirumab); or
for subjects selected not to receive the EGFR-directed therapeutic agent, administering a therapy course that does not comprise the EGFR-directed therapeutic agent (such as a drug that targets VEGF), FOLFOX (optionally in combination with a drug that targets VEGF), FOLFIRI (optionally in combination with a drug that targets VEGF), CapeOX (optionally in combination with a drug that targets VEGF), FOLFOXIRI (optionally in combination with a drug that targets VEGF), 5-FU and leucovorin (optionally in combination with a drug that targets VEGF), Capecitabine (optionally in combination with a drug that targets VEGF), Irinotecan (optionally in combination with a drug that targets VEGF), Regorafenib, or Trifluridine and tipiracil (optionally in combination with a drug that targets VEGF)).

### V. Examples

A. Patients, Materials, and Methods

460 KRAS c.12,13,59-61-wt patients were previously randomized to second-line irinotecan (Ir) versus (vs) irinotecan with panitumumab (IrPan). See Seymour et al. and Middleton et al. The current study includes all consenting patients with adequate stored pathological material (326 of 460 [70.9%] patients) (tissue collected at trial entry) where IHC for AREG and EREG was successful (n=313). There were 13 cases in which IHC failed and insufficient tumor tissue was available for repeat analysis. Additional mutational analysis for KRAS c.146, NRAS c.12,13,59-61, BRAF c.1799T>A was previously performed. The primary analysis was conducted in patients who were KRAS c.12,13,59-61,146 and NRAS c.12,13,59-61 wt (“RAS-wt”).

Three 4 μm thick tissue sections from each FFPE block were cut onto separate Superfrost Plus slides (VWR, Lutterworth, UK). One section was stained with anti-AREG (SP272) (Ventana Medical Systems, Inc., Tucson, Ariz., USA) and another with anti-EREG (SP326) (Ventana Medical Systems, Inc. Tucson, Ariz., USA) rabbit monoclonal antibodies using a VENTANA BenchMark ULTRA autostainer (Ventana Medical Systems, Inc. Tucson, Ariz., USA) and pre-programmed protocols. The third section was stained with hematoxylin and eosin (H&E) using Mayer's hematoxylin and Scott's tap water substitute as the blueing reagent. Digital images of slides were generated using a VENTANA DP200 scanner at 200 times magnification. The researchers were blinded to patients' treatment allocations, mutation profiles, and clinical outcomes.

AI algorithms to evaluate the percentage of tumor cells staining positive for AREG and EREG were developed by Ventana Medical Systems, Inc. (Santa Clara, Calif., USA; Tucson, Ariz., USA). To verify the algorithms, 30 fields of view (FOV) within the tumor area were initially selected at random from 30 digital slide images generated using an in-house development cohort of colorectal cancer specimens stained for AREG and EREG. Two pathologists independently annotated all tumor cells within the FOVs as positive or negative for each of AREG and EREG. Four staining patterns were observed: membranous, cytoplasmic, cytomembranous and punctate. A tumor cell was regarded as positive if any one of these staining patterns was seen. Inter-pathologist agreement and agreement between algorithm- and pathologist-generated results were assessed using the concordance correlation coefficient (CCC). To verify the algorithms when used to analyze whole-slide images, the AI algorithm IHC read outs were correlated to the qRT-PCR results within the tumor areas from the PICCOLO cohort.

Tumor areas were annotated by pathologists and the developed AI algorithms were applied to the whole slides, and then data extracted from the tumor areas. The AI algorithms determined the percentage of IHC positive tumor cells for each of AREG and EREG. The samples were dichotomized into high (either AREG high or EREG high) and low (both AREG and EREG low) expressors by IHC percentage positivity. A receiver operating characteristic (ROC) curve was generated for all possible combinations of AREG and EREG IHC cut points (1% to 100% for each, therefore 1002=10,000 combinations), referring to a previous mRNA dichotomisation as reported by Seligmann et al. as the basis for sensitivity and specificity calculations. The maximal Youden Index (Max_{c} [sensitivity_{c}+specificity_{c}−1], i.e. where the sum of sensitivity and specificity was at its maximum when equal weighting was placed on each) was calculated to identify the IHC cut point that best aligned with that of the mRNA assay. See Youden. This was taken to define the IHC cut point for investigation in the primary analysis.

The primary endpoint was progression-free survival (PFS); secondary endpoints were overall survival (OS) and Response Evaluation Criteria In Solid Tumors (RECIST) response rate (RR). PFS and RR data were unchanged from the primary PICCOLO trial analysis but updated 2-year OS data were used in this analysis.

Stata was used for all statistical analyses (Stata Statistical Software, Release 16 [2019]; StataCorp). Baseline patient characteristics were compared between treatment arms (IrPan vs Ir) using 2-tailed t tests, Wilcoxon rank sum tests (for variables with non-normally distributed frequency distributions), and Pearson χ2 tests (for categorical variables). Patient characteristics were compared with the whole trial population using the same tests. Box-plots and Wilcoxon rank sum tests were used to compare the distributions of continuous AREG and EREG IHC percentage positivity between BRAF-wt and mutant cases, left (splenic flexure to rectum) vs right PTL, and finally presence vs absence of peritoneal metastases.

Ligand expression (i.e., IHC percentage positivity) was first assessed as a prognostic marker in all patients treated with Ir alone—using both the dichotomous classifier (high vs low) and each ligand separately as a continuous variable—in Cox proportional hazards models. Where AREG and EREG were assessed as continuous variables, percentage ligand positivity was scaled down by a factor of 10 to enhance the interpretability of hazard ratios (HR). Analyses were first performed unadjusted and then adjusted for World Health Organization performance status [WHO PS], response to previous therapy, and previous chemotherapy (yes vs no). Response to previous therapy was unknown in 30 patients and multiple imputation was used to impute values for these 30 patients. Multiple logistic regression was performed using “previous oxaliplatin therapy” and “previous chemotherapy” as predictors of previous response based on 20 imputed data sets.

Ligand expression was then assessed as a predictive marker for panitumumab therapy benefit on PFS and OS in unadjusted Cox proportional hazards models stratifying by IHC ligand status (either AREG or EREG high vs both AREG and EREG low) and assessing treatment effects (IrPan vs Ir) and testing for ligand-treatment interactions using likelihood ratio tests. These models were then repeated adjusting separately for BRAF mutation, PTL and the presence of peritoneal metastases to determine whether the ligand-treatment interactions persisted after adjustment for these possible confounding factors. The unadjusted models were also repeated in the RAS and BRAF-wt group and also considering dichotomous AREG and dichotomous EREG separately (50% cut points). Kaplan-Meier (KM) curves were plotted. The concordance probability was assessed using Harrell's C index.

Unadjusted risk ratios for the effect of IrPan vs Ir on RR were estimated from generalized linear models (with a log link) stratified by dichotomous ligand IHC. The likelihood ratio test for ligand-treatment interaction was then performed.

B. Results

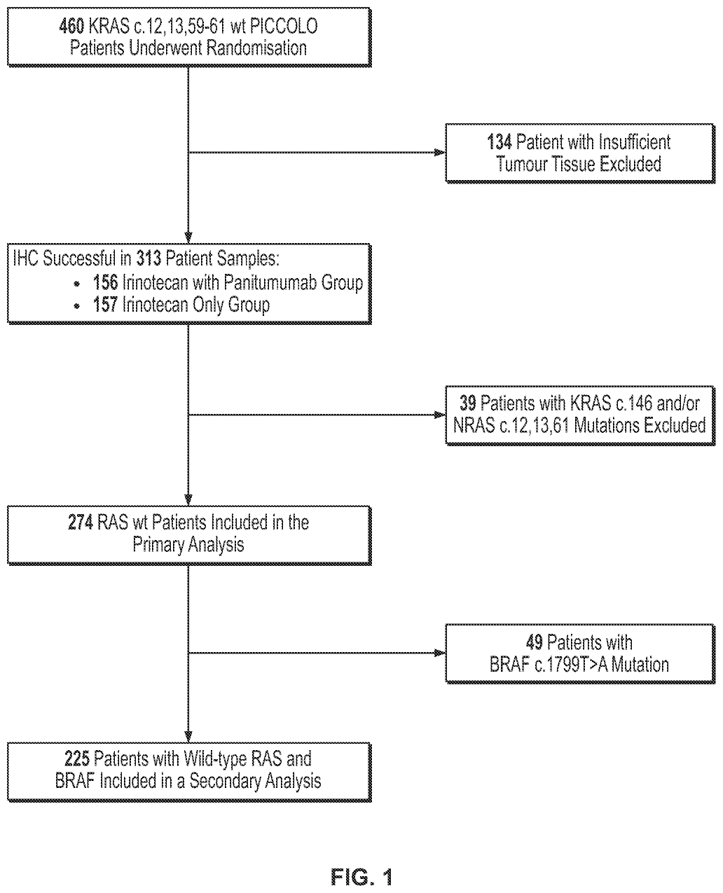

There were no significant differences in characteristics between the patient cohort in this biomarker study and the main trial population (data not shown). A total of 274 of 313 (88%) patients were RAS-wt, of which 49 (18%) had a BRAF mutation (FIG. 1). 242 (88%) RAS-wt patients had a disease progression event and 248 (91%) had died.

B1. Algorithm Development

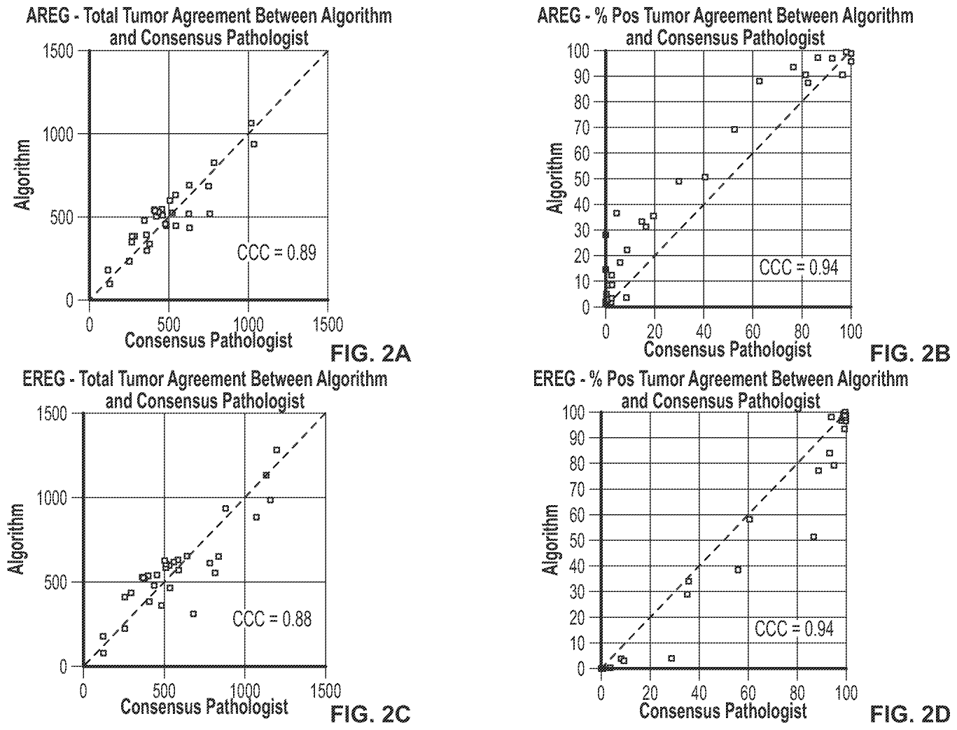

There was strong agreement between the two independent pathologists for assessing total tumor cell count and AREG and EREG IHC percentage positivity in each of the 30 FOVs analyzed. Each pathologist's scores were also highly correlated with the AI algorithms' assessments (FIG. 2).

B2. Ligand Expression

AREG and EREG IHC percentage positivity were strongly correlated (Spearman correlation coefficient 0.77, P<0.0005). Among the subset of patients for whom both IHC and mRNA data were available (186 of 313 [59%] patients), the two measures were positively correlated for each of the ligands (Spearman correlation coefficient: AREG 0.64, P<0.0001; EREG 0.80, P<0.0001).

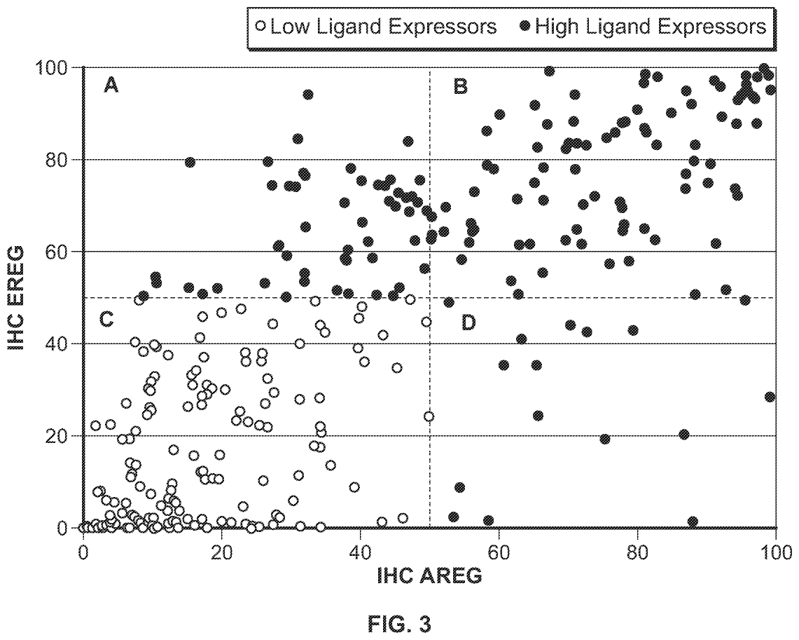

For the primary analysis, AREG and EREG IHC percentage positivity were assessed a priori as a dichotomous marker (high expression of either ligand vs low expression of both), to aid the route to clinical application. Where both IHC and mRNA data were available, the maximal Youden Index was 0.581, which determined “high” vs “low” cut points of 47% IHC positivity for AREG and 52% for EREG. Sensitivity and specificity with these cut points were 0.716 and 0.865 respectively. Use of these cut points divided the study sample (n=313) into 159 (50.8%) high and 154 (49.2%) low cases. To facilitate manual AREG and EREG interpretation in future clinical practice, the optimal cut points were rounded to 50% for both markers in analyses going forwards. A scatter plot of EREG IHC percentage positivity vs AREG IHC percentage positivity is shown in FIG. 3 with dotted lines depicting the 50% cut points. The high and low ligand groups had an even distribution of patients in each treatment group. There were significantly more patients with right-sided PTL, BRAF-mutant status and peritoneal metastases in the low than the high ligand group (Table 6).

TABLE 6
Descriptive statistics of characteristics of RAS-wt patients in low
and high ligand expression groups and p-values for association

| | | Low ligand | High ligand | p-value |
|---|---|---|---|---|
| | | expression | expression | comparing |
| | | (≤50% | (>50% | low and |
| | | AREG & | AREG or | high |
| Patient | | ≤50% EREG) | >50% EREG) | ligand |
| characteristic | Category | (n = 142) | (n = 132) | expression* |
| Treatment | Ir | 74 (52.1) | 66 (50.0) | 0.73 |
| arm N(%) | IrPan | 68 (47.9) | 66 (50.0) | |
| Age at | | Mean 61.8 | Mean 61.9 | 0.86 |
| randomization | | (s.d 10.3) | (s.d 10.3) | |
| (yrs) | | | | |
| Sex N(%) | Male | 93 (65.5) | 98 (74.2) | 0.14 |
| | Female | 48 (33.8) | 34 (25.8) | |
| | Unknown | 1 (0.7) | 0 (0) | |
| Primary | Left | 76 (53.5) | 112 (84.9) | <0.0005 |
| tumor location | (including | | | |
| (PTL) N(%) | rectal) | | | |
| | Right | 64 (45.1) | 18 (13.6) | |
| | Unknown | 2 (1.4) | 2 (1.5) | |
| Peritoneal | No | 97 (68.3) | 110 (83.3) | 0.001 |
| metastases | Yes | 42 (29.6) | 17 (12.9) | |
| N(%) | Unknown | 3 (2.1) | 5 (3.8) | |
| BRAF_{c.1799T>A} | Wild-type | 98 (69.0) | 127 (96.2) | <0.0005 |
| N(%) | Mutant | 44 (31.0) | 5 (3.8) | |
| Performance | 0-1 | 134 (94.4) | 125 (94.7) | 0.90 |
| status N(%) | 2 | 8 (5.6) | 7 (5.3) | |
| mRNA ligand data | No | 41 (28.9) | 65 (49.2) | 0.001 |
| available N(%) | Yes | 101 (71.1) | 67 (50.8) | |
| Resection or | Resection | 104 (73.2) | 67 (50.8) | <0.0005 |
| biopsy N(%) | Biopsy | 38 (26.8) | 65 (49.2) | |
| Overall survival | | Median 9.3 | Median 11.5 | 0.24** |
| time (months) | | (IQR | (IQR | |
| | | 4.3-19.2) | 7.9-20.0) | |
| Death event N(%) | No | 12 (8.5) | 14 (10.6) | 0.54 |
| | Yes | 130 (91.5) | 118 (89.4) | |
| Progression | | Median 3.8 | Median 5.5 | 0.56** |
| free survival | | (IQR 2.7-8.1) | (IQR 2.8-9.2) | |
| time (months) | | | | |
| Progression | No | 15 (10.6) | 17 (12.9) | 0.55 |
| event N(%) | Yes | 127 (89.4) | 115 (87.1) | |
| Best response | CR or PR | 27 (19.0) | 35 (26.5) | 0.12 |
| N(%) | SD or PD | 113 (79.6) | 93 (70.5) | |
| | Unknown | 2 (1.4) | 4 (3.0) | |

*Unknown category excluded from tests. Wilcoxon rank sum test used for age and Pearson Chi-squared test or Fisher's exact test used for categorical variables
**p-value from log rank test for equality of survivor functions

B3. AREG/EREG Performance as a Combined Dichotomous Biomarker

There was no evidence for a prognostic effect of IHC ligand status (high vs low) on PFS (unadjusted HR, 1.22 [95% CI, 0.86-1.75]; P=0.27) or OS (unadjusted HR, 1.01 [95% CI, 0.71-1.43]; P=0.98) in patients treated with Ir alone, or in adjusted analyses (Table 7).

TABLE 7
Prognostic analysis for the effect of the dichotomous classifier
(50% IHC positivity cut point) on PFS and OS in RAS-wt patients
treated with Ir alone (unadjusted and adjusted HRs and 95% CIs)

| | | | Ir | | Ir | |
|---|---|---|---|---|---|---|
| | Dichotomous | Events/ | Unadjusted | | Adjusted | |
| Outcome | classifier | patients | HR (95% CI) | p-value | HR (95% CI)* | p-value |
| PFS | AREG EREG: | 125/136 | 1.22 | 0.27 | 1.11 | 0.57 |
| | high vs low | | (0.86-1.75) | | (0.77-1.62) | |
| OS | AREG EREG: | 127/140 | 1.01 | 0.98 | 0.93 | 0.70 |
| | high vs low | | (0.71-1.43) | | (0.65-1.34) | |

*Adjusted for WHO PS, previous response and previous chemotherapy (yes vs no).

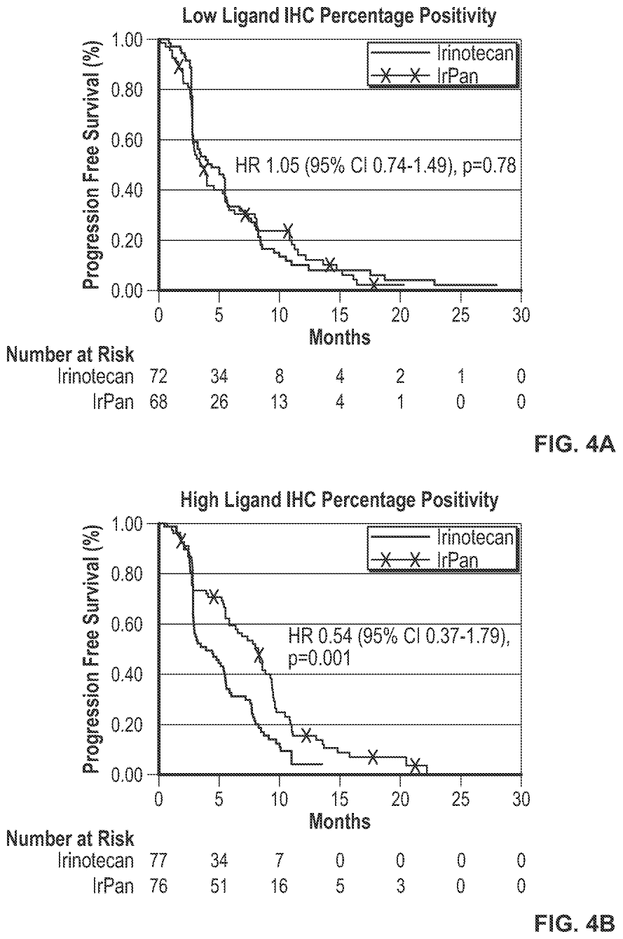

The primary hypothesis was that high ligand IHC score would be predictive of PFS benefit of panitumumab in RAS-wt patients. This hypothesis was supported by the data, as shown in Table 8 and FIG. 4.

TABLE 8
Estimated crude HRs for the effect of treatment (IrPan vs Ir) on PFS and OS in RAS-wt patients, then RAS-wt and BRAF-wt
patients, stratified by the dichotomous classifier and including likelihood ratio tests for ligand-treatment interaction

| | | | | Low ligand expression | | High ligand expression | | p-value for |
|---|---|---|---|---|---|---|---|---|
| | | All patients | | (≤50% AREG & ≤50% EREG) | | (>50% AREG or >50% EREG) | | ligand- |
| | Mutation | Events/ | Unadjusted | Events/ | Unadjusted | Events/ | Unadjusted | treatment |
| Outcome | subgroup | patients | HR (95% CI) | patients | HR (95% CI) | patients | HR (95% CI) | interaction |
| PFS | RAS-wt | 242/269 | 0.77 | 127/140 | 1.05 | 115/129 | 0.54 | 0.02 |
| | | | (0.60-1.00) | | (0.74-1.49) | | (0.37-0.79) | |
| | | | p = 0.05 | | p = 0.78 | | p = 0.001 | |
| | RAS-wt | 197/221 | 0.65 | 87/97 | 0.80 | 110/124 | 0.53 | 0.21 |
| | and | | (0.49-0.86) | | (0.52-1.23) | | (0.36-0.78) | |
| | BRAF-wt | | p = 0.003 | | p = 0.31 | | p = 0.001 | |
| OS | RAS-wt | 248/274 | 1.03 | 130/142 | 1.21 | 118/132 | 0.87 | 0.19 |
| | | | (0.80-1.32) | | (0.86-1.71) | | (0.60-1.25) | |
| | | | p = 0.81 | | p = 0.28 | | p = 0.44 | |
| | RAS-wt | 202/225 | 0.90 | 89/98 | 0.95 | 113/127 | 0.84 | 0.65 |
| | and | | (0.68-1.18) | | (0.62-1.45) | | (0.58-1.21) | |
| | BRAF-wt | | p = 0.44 | | p = 0.81 | | p = 0.34 | |

In RAS-wt patients with high ligand IHC positivity, median PFS with IrPan was 8.0 months, compared with 3.2 months with Ir alone (HR, 0.54 [95% CI, 0.37-0.79]; P=0.001). Conversely, there no was benefit from panitumumab therapy in patients with low ligand IHC positivity (median PFS IrPan vs Ir: 3.4 vs 4.4 months; HR, 1.05 [95% CI, 0.74-1.49]; p=0.78) (Table 8, FIG. 4). The ligand-treatment interaction was significant whether unadjusted (p=0.02) or adjusted (p=0.02). The results for OS were less marked (interaction p=0.19), most likely due to the lack of OS effect of panitumumab overall in the trial. Within the RAS-wt and BRAF-wt subpopulation, the effect sizes were similar, though less significant likely due to smaller sample size (PFS interaction p=0.21) (Table 8).

In RAS-wt patients with high ligand IHC positivity, the RR was significantly improved by the addition of panitumumab to irinotecan (IrPan vs Ir: 48% vs 6%; risk ratio, 7.8 [95% CI, 2.90-20.69]; p<0.0001) whereas in patients with low ligand IHC positivity the RR was not significantly different between the two treatment groups (IrPan vs Ir: 25% vs 14%; risk ratio, 1.8 [95% CI, 0.89-3.65]; p=0.10) (Table 9).

TABLE 9
Estimated crude risk ratios and 95% CIs for the effect of treatment arm on the risk of
complete or partial response in RAS-wt patients stratified by the ligand dichotomous
classifier and including the likelihood ratio test for ligand-treatment interaction.

| | Low ligand expression | | | | High ligand expression | | | | |
|---|---|---|---|---|---|---|---|---|---|
| | (≤50% AREG & ≤50% EREG) | | | | (>50% AREG or >50% EREG) | | | | |
| | Complete | | | | Complete | | | | |
| | or partial | | | | or partial | | | | p-value for |
| | response | | | | response | | | | ligand- |
| | n(%) | | Risk ratio | | n(%) | | Risk ratio | | treatment |
| Treatment | No | Yes | (95% CI) | p-value | No | Yes | (95% CI) | p-value | interaction |
| Ir | 62 | 10 | 1.0 | | 60 | 4 | 1.0 | | |
| | (86.1) | (13.9) | | | (93.7) | (6.3) | | | |
| IrPan | 51 | 17 | 1.8 | 0.10 | 33 | 31 | 7.8 | <0.0001 | 0.01 |
| | (75.0) | (25.0) | (0.89-3.65) | | (51.6) | (48.4) | (2.90-20.69) | | |

B4. AREG and EREG as Separate Biomarkers

As a secondary analysis, AREG and EREG IHC percentage positivity were examined separately as continuous variables. As in the primary analysis of the combined dichotomous classifier, neither AREG nor EREG were prognostic for PFS or OS as continuous variables (Table 10). EREG was predictive of PFS benefit from panitumumab (interaction p=0.01). Although a trend towards the same finding was seen for AREG, the ligand-treatment interaction did not reach significance (interaction p=0.06). Neither AREG nor EREG were predictive of panitumumab therapy OS benefit (Table 11).

TABLE 10
Prognostic analysis for the effect of AREG and EREG as continuous variables
(scaled by a factor of 10) on PFS and OS in RAS-wt patients treated
with Ir alone (unadjusted and adjusted HRs and 95% CIs).

| | | | Ir | | Ir | |
|---|---|---|---|---|---|---|
| | Ligand | Events/ | Unadjusted | | Adjusted | |
| Outcome | (continuous) | patients | HR (95% CI) | p-value | HR (95% CI)* | p-value |
| PFS | AREG | 125/136 | 1.04 | 0.27 | 1.01 | 0.86 |
| | | | (0.97-1.10) | | (0.94-1.08) | |
| | EREG | 124/135 | 1.02 | 0.57 | 1.00 | 0.91 |
| | | | (0.96-1.07) | | (0.95-1.06) | |
| OS | AREG | 127/140 | 0.98 | 0.53 | 0.94 | 0.12 |
| | | | (0.92-1.05) | | (0.88-1.01) | |
| | EREG | 126/139 | 0.96 | 0.19 | 0.95 | 0.07 |
| | | | (0.91-1.02) | | (0.89-1.01) | |

*Adjusted for WHO PS, previous chemotherapy and previous response to chemotherapy.

TABLE 11
Estimated crude HRs and 95% CIs for the effect of continuous AREG and EREG (scaled
by a factor of 10) on PFS and OS in RAS-wt, then RAS-wt and BRAF-wt patients.
HRs are shown for all patients, then the Ir group and finally the IrPan group.
Likelihood ratio tests for ligand-treatment interaction are shown.

| | | | | | | p-value for |
|---|---|---|---|---|---|---|
| | | | All patients | Ir | IrPan | ligand- |
| | Mutation | Ligand | Unadjusted | Unadjusted | Unadjusted | treatment |
| Outcome | subgroup | (continuous) | HR (95% CI) | HR (95% CI) | HR (95% CI) | interaction |
| PFS | RAS-wt | AREG | 0.99 | 1.04 | 0.95 | 0.06 |
| | | | (0.95-1.04), | (0.97-1.10), | (0.89-1.02), | |
| | | | p = 0.74 | p = 0.27 | p = 0.18 | |
| | | EREG | 0.97 | 1.02 | 0.93 | 0.01 |
| | | | (0.93-1.01), | (0.96-1.07), | (0.87-0.98), | |
| | | | p = 0.15 | p = 0.57 | p = 0.009 | |
| | RAS-wt | AREG | 1.02 | 1.04 | 1.01 | 0.38 |
| | and | | (0.97-1.07), | (0.97-1.12), | (0.94-1.09), | |
| | BRAF-wt | | p = 0.41 | p = 0.22 | p = 0.81 | |
| | | EREG | 1.00 | 1.02 | 0.98 | 0.23 |
| | | | (0.95-1.05), | (0.96-1.09), | (0.91-1.04), | |
| | | | p = 0.96 | p = 0.48 | p = 0.49 | |
| OS | RAS-wt | AREG | 0.96 | 0.98 | 0.94 | 0.43 |
| | | | (0.92-1.01), | (0.92-1.05), | (0.88-1.01), | |
| | | | p = 0.12 | p = 0.53 | p = 0.09 | |
| | | EREG | 0.93 | 0.96 | 0.91 | 0.17 |
| | | | (0.90-0.97), | (0.91-1.02), | (0.86-0.96), | |
| | | | p = 0.001 | p = 0.19 | p = 0.001 | |
| | RAS-wt | AREG | 1.00 | 1.00 | 1.00 | 0.98 |
| | and | | (0.95-1.05), | (0.93-1.07), | (0.93-1.08), | |
| | BRAF-wt | | p = 0.98 | p = 0.98 | p = 0.95 | |
| | | EREG | 0.97 | 0.98 | 0.96 | 0.63 |
| | | | (0.93-1.02), | (0.92-1.05), | (0.90-1.03), | |
| | | | p = 0.21 | p = 0.61 | p = 0.24 | |

B5. Effect of Possible Confounding Factors

Given that BRAF mutation, right-sided PTL and the presence of peritoneal metastases were associated with low ligand IHC positivity, whether the differential treatment effects by ligand level seen for PFS in the RAS-wt patients were instead driven by these factors was investigated (Table 12). The dichotomous classifier remained a significant predictor of panitumumab therapy benefit after adjustment for BRAF (interaction P=0.02), PTL (interaction P=0.01), and peritoneal metastases (interaction P=0.01). Similar results were found when each ligand was examined separately as a continuous variable. Thus, the ligand-treatment effect appears to be independent of these potential confounders.

TABLE 12
HRs and 95% CIs for the effect of continuous AREG, continuous EREG and the
dichotomous classifier (high vs low) on PFS in RAS-wt patients adjusted for BRAF (mutant vs
wt), PTL (right vs left) and peritoneal metastases (present vs absent) stratified by treatment arm
and including likelihood ratio tests for ligand-treatment interaction

| | | | | p-value for |
|---|---|---|---|---|
| | All patients | Ir | IrPan | ligand- |
| | Adjusted | Adjusted | Adjusted HR | treatment |
| HRs for PFS | HR (95% CI) | HR (95% CI) | (95% CI) | interaction |
| AREG: continuous | 1.02 (0.97- | 1.04 (0.98-1.12), | 0.99 (0.92-1.07), | 0.04 |
| BRAF mutant | 1.07), p = 0.48 | p = 0.20 | p = 0.89 | |
| | 1.84 (1.30- | 1.22 (0.72-2.08), | 2.82 (1.75-4.56), | |
| | 2.61), p = 0.001 | p = 0.47 | p < 0.0005 | |
| EREG: continuous | 0.99 (0.95- | 1.02 (0.96-1.09), | 0.97 (0.91-1.03), | 0.01 |
| BRAF mutant | 1.04), p = 0.79 | p = 0.46 | p = 0.29 | |
| | 1.73 (1.21- | 1.18 (0.69-2.01), | 2.50 (1.53-4.10), | |
| | 2.47), p = 0.003 | p = 0.56 | p < 0.0005 | |
| AREG EREG: high | 1.05 (0.80- | 1.27 (0.87-1.84), | 0.88 (0.59-1.31), | 0.02 |
| vs low | 1.38), p = 0.72 | p = 0.21 | p = 0.52 | |
| BRAF mutant | 1.80 (1.27- | 1.20 (0.71-2.03), | 2.72 (1.69-4.38), | |
| | 2.54), p = 0.001 | p = 0.49 | p < 0.0005 | |
| AREG: continuous | 1.00 (0.95- | 1.04 (0.97-1.11), | 0.96 (0.89-1.03), | 0.05 |
| PTL (right vs left | 1.05), p = 0.88 | p = 0.29 | p = 0.28 | |
| [incl. rectum]) | 1.10 (0.83- | 0.97 (0.66-1.42), | 1.20 (0.77-1.87), | |
| | 1.47), p = 0.50 | p = 0.87 | p = 0.41 | |
| EREG: continuous | 0.97 (0.93- | 1.02 (0.96-1.08), | 0.92 (0.87-0.99), | 0.008 |
| PTL (R vs L) | 1.02), p = 0.21 | p = 0.52 | p = 0.02 | |
| | 1.04 (0.77- | 0.96 (0.65-1.41), | 1.01 (0.64-1.61), | |
| | 1.39), p = 0.80 | p = 0.83 | p = 0.95 | |
| AREG EREG: high | 0.96 (0.73- | 1.27 (0.87-1.85), | 0.73 (0.48-1.11), | 0.01 |
| vs low | 1.28), p = 0.79 | p = 0.23 | p = 0.14 | |
| PTL (R vs L) | 1.09 (0.81- | 1.00 (0.67-1.47), | 1.10 (0.69-1.76), | |
| | 1.48), p = 0.56 | p = 0.99 | p = 0.69 | |
| AREG: continuous | 1.00 (0.95- | 1.06 (0.99-1.13), | 0.95 (0.88-1.02), | 0.03 |
| Peritoneal mets (yes | 1.05), p = 0.95 | p = 0.10 | p = 0.15 | |
| vs no) | 1.30 (0.94- | 1.34 (0.90-2.01), | 1.07 (0.62-1.85), | |
| | 1.79), p = 0.11 | p = 0.15 | p = 0.81 | |
| EREG: continuous | 0.98 (0.94- | 1.03 (0.97-1.09), | 0.92 (0.86-0.98), | 0.009 |
| Peritoneal mets (yes | 1.24 (0.90- | 1.31 (0.88-1.96), | 0.97 (0.57-1.64), | |
| vs no) | 1.70), p = 0.19 | p = 0.18 | p = 0.90 | |
| | 1.02), p = 0.32 | p = 0.33 | p = 0.01 | |
| AREG EREG: high | 0.99 (0.76- | 1.38 (0.96-2.00), | 0.70 (0.48-1.04), | 0.01 |
| vs low | 1.29), p = 0.95 | p = 0.09 | p = 0.08 | |
| Peritoneal mets (yes | 1.29 (0.94- | 1.35 (0.90-2.01), | 1.03 (0.59-1.78), | |
| vs no) | 1.78), p = 0.12 | p = 0.15 | p = 0.93 | |

Adenocarcinomas that originate from the right side of the colon (caecum to splenic flexure) are more frequently associated with BRAF, PTEN and PIK3CA mutations, as well as mismatch repair enzyme deficiencies. The relationship between tumor sidedness—as a proxy for such molecular characteristics—and anti-EGFR therapy has been extensively examined, with retrospective analyses of the CRYSTAL (FOLFIRI+/−cetuximab) and PRIME (FOLFOX+/−panitumumab) trials demonstrating OS benefit from anti-EGFR agents in RAS-wt patients with left-but not right-sided PTL. These data have been used to inform national treatment guidelines, with the NCCN recommending that right-sided PTL patients should not be offered anti-EGFR agents in the first-line setting. Here, AREG and EREG protein expression were higher with left-rather than right-sided PTL. However, our dichotomous classifier remained a significant predictor of panitumumab benefit after adjustment for this factor, indicating its potential clinical utility in identifying right PTL patients who could be appropriately offered anti-EGFR therapy early in their treatment course.

B6. Interrogation of Combined AREG/EREG Model—Alternative Cut Points

A 50% IHC positivity cut point was chosen as it closely aligned with the middle/upper tertile cut point developed in the previously validated mRNA expression-based assay. For exploratory purposes, panitumumab benefit on PFS using different cut points was examined (Table 13). Lowering the cut point to 20% identified patients with low ligand IHC percentage positivity who in fact had significantly inferior PFS with the addition of panitumumab (HR, 1.73 [95% CI, 1.02-2.95]; p=0.04) and were perhaps harmed by this treatment. In the high ligand group, the HRs for IrPan vs Ir on PFS showed marked benefit with HRs around 0.55 for the 20% up to the 50% cut point. The ligand-treatment interaction remained significant throughout this range, indicating potential for the use of different cut points in different clinical scenarios. Additionally, when the cut point was lowered to 20%, a group of very low ligand expressors was identified who appeared to be harmed by the addition of panitumumab to chemotherapy.

TABLE 13
Estimated crude HRs and 95% CIs for the effect of treatment arm
(IrPan vs Ir) on PFS in RAS-wt patients stratified by ligand
percentage positivity at various cut points and
including the likelihood ratio tests for ligand-treatment interactions

| | | | | | P-value |
|---|---|---|---|---|---|
| | | | | | for |
| Cut | | | | | ligand- |
| point | | | | | treat- |
| Low | Low ligand expression | | High ligand expression | | ment |
| vs | Events/ | Unadjusted HR | Events/ | Unadjusted HR | inter- |
| High | patients | (95% CI) | patients | (95% CI) | action |
| 20% | 57/63 | 1.73 (1.02-2.95), | 185/206 | 0.54 (0.40-0.73), | 0.0001 |
| | | p = 0.04 | | p = 0.0001 | |
| 25% | 68/74 | 1.40 (0.86-2.26), | 174/195 | 0.57 (0.42-0.78), | 0.005 |
| | | p = 0.17 | | p = 0.0004 | |
| 30% | 85/91 | 1.23 (0.80-1.88), | 157/178 | 0.58 (0.42-0.80), | 0.02 |
| | | p = 0.35 | | p = 0.001 | |
| 40% | 111/122 | 1.12 (0.77-1.63), | 131/147 | 0.55 (0.39-0.79), | 0.02 |
| | | p = 0.56 | | p = 0.001 | |
| 50% | 127/140 | 1.05 (0.74-1.49), | 115/129 | 0.54 (0.37-0.79), | 0.02 |
| | | p = 0.78 | | p = 0.001 | |
| 60% | 147/162 | 0.95 (0.68-1.32), | 94/106 | 0.61 (0.40-0.92), | 0.13 |
| | | p = 0.75 | | p = 0.02 | |
| 70% | 165/184 | 0.86 (0.63-1.18), | 75/83 | 0.64 (0.40-1.02), | 0.27 |
| | | p = 0.36 | | p = 0.06 | |
| 75% | 180/202 | 0.84 (0.63-1.13), | 60/65 | 0.61 (0.36-1.04), | 0.38 |
| | | p = 0.25 | | p = 0.07 | |
| 80% | 194/218 | 0.86 (0.65-1.14), | 46/49 | 0.45 (0.24-0.83), | 0.10 |
| | | p = 0.30 | | p = 0.01 | |
| 90% | 214/239 | 0.82 (0.62-1.07), | 26/28 | 0.53 (0.24-1.19), | 0.28 |
| | | p = 0.14 | | p = 0.12 | |

B7. Comparison of Biopsies and Resections

Within the study sample, 190 (61%) tumor specimens were taken from resections and 123 (39.3%) from biopsies. To ensure our dichotomous model remained reliable irrespective of the tumor specimen type examined, we also analysed these subgroups separately (Table 14). The HR for the effect of treatment (IrPan vs Ir) on PFS favored the addition of panitumumab for patients who had undergone resections (HR, 0.62 [95% CI, 0.44-0.86]; p=0.005) but not those with biopsies only (HR, 1.06 [95% CI, 0.70-1.62]; p=0.78), perhaps because these patients were more likely to have presented with widely metastatic disease rather than recurrence of disease detected during routine post-operative surveillance. That said, among those with biopsies only there remained a trend to suggest the addition of panitumumab to irinotecan might benefit high ligand expressors and not low ligand expressors. The ligand-treatment interactions did not reach significance for either specimen type, likely due to the smaller sample sizes in this subgroup analysis.

TABLE 14
Estimated crude HRs for the effect of treatment (IrPan vs Ir) on PFS in RAS-wt patients with resection or biopsy specimens
stratified by the dichotomous classifier and including likelihood ratio tests for ligand-treatment interaction.

| | | | | Low ligand expression | | High ligand expression | | p-value for |
|---|---|---|---|---|---|---|---|---|
| | | All patients | | (≤50% AREG & ≤50% EREG) | | (>50% AREG or >50% EREG) | | ligand- |
| | Specimen | Events/ | Unadjusted | Events/ | Unadjusted | Events/ | Unadjusted | treatment |
| Outcome | type | patients | HR (95% CI) | patients | HR (95% CI) | patients | HR (95% CI) | interaction |
| PFS | Resection | 153/170 | 0.62 | 91/103 | 0.83 | 62/67 | 0.38 | 0.06 |
| | | | (0.44-0.86), | | (0.54-1.27), | | (0.22-0.66), | |
| | | | p = 0.005 | | p = 0.39 | | p = 0.001 | |
| | Biopsy | 89/99 | 1.06 | 36/37 | 1.64 | 53/62 | 0.78 | 0.07 |
| | | | (0.70-1.62), | | (0.83-3.25), | | (0.45-1.35), | |
| | | | p = 0.78 | | p = 0.15 | | p = 0.38 | |

### REFERENCES

Bokemeyer et al., Addition of cetuximab to chemotherapy as first-line treatment for KRAS wild-type metastatic colorectal cancer: Pooled analysis of the CRYSTAL and OPUS randomized clinical trials. Eur J Cancer. (2012), 48(10), pp. 1466-1475.
Douillard et al., Panitumumab-FOLFOX4 treatment and RAS mutations in colorectal cancer, N Engl J Med. (2013), 369(11), pp. 1023-1034.
Karapetis et al., K-ras mutations and benefit from cetuximab in advanced colorectal cancer. N Engl J Med. (2008), 359(17), pp. 1757-1765.
Kheiwatty et al., Oncotarget (January, 31, 17); Vol. 8, Issue 5, pp, 7666-7677.
Middleton et al., A randomized phase III trial of the pharmacokinetic biomodulation of irinotecan using oral ciclosporin in advanced colorectal cancer: Results of the Panitumumab, Irinotecan &amp; Ciclosporin in COLOrectal cancer therapy trial (PICCOLO). Eur J Cancer (2013), Vol. 49, Issue 16, pp. 3507-3516.
Perkins et al., Beyond KRAS status and response to anti-EGFR therapy in metastatic colorectal cancer, Pharmacogenetics, Vol. 15, Issue 7, pp. 1043-52 (2014).
Prior et al., A Comprehensive Survey of Ras Mutations in Cancer, Cancer Res. (May 2012), Vol. 72, Issue 10, pp. 2457-67.
Seymour et al., Panitumumab and irinotecan versus irinotecan alone for patients with KRAS wild-type, fluorouracil-resistant advanced colorectal cancer (PICCOLO): a prospectively stratified randomised trial. Lancet Oncol. (2013) Vol. 14, Issue 8, pp. 749-759.
Tejpar et al., Prognostic and Predictive Relevance of Primary Tumor Location in Patients With RAS Wild-Type Metastatic Colorectal Cancer, JAMA Oncol. (2017), 3(2), p. 194.
Vale et al. Does anti-EGFR therapy improve outcome in advanced colorectal cancer? A systematic review and meta-analysis. Cancer Treat Rev. 2012; 38(6), pp. 618-625.
Van Cutsem et al. Fluorouracil, leucovorin, and irinotecan plus cetuximab treatment and RAS mutations in colorectal cancer, J Clin Oncol. (2015), 33(7), pp. 692-700.
Waring et al., RAS Mutations as Predictive Biomarkers in Clinical Management of Metastatic Colorectal Cancer, Clinical Colorectal Cancer (June 2016), Vol. 15, Issue 2, pp. 95-103.
Yoshida et al., A novel predictive strategy by immunohistochemical analysis of four EGFR ligands in metastatic colorectal cancer treated with anti-EGFR antibodies, Journal of Cancer Research and Clinical Oncology (March 2013), Volume 139, Issue 3, pp 367-378.
Youden, Index for rating diagnostic tests, Cancer (1950), Vol. 3, Issue 1, pp. 32-35.

### ADDITIONAL EMBODIMENTS

Additional Embodiment 1. A method of treating a patient with a tumor, the method comprising administering to the patient a therapeutic course that includes an EGFR-directed therapeutic agent if the tumor is either AREG HIGH or EREG HIGH, wherein the tumor is considered AREG HIGH if the tumor has been histochemically or cytochemically determined to have a percentage of AREG+ tumor cells that is greater than or equal to a first pre-determined cut off and wherein the tumor is considered EREG HIGH if the tumor has been histochemically or cytochemically determined to have a percentage of EREG+ tumor cells that is greater than or equal to a second pre-determined cut off.

Additional Embodiment 2. A method of treating a patient with a tumor, the method comprising administering a treatment to the patient a therapeutic course that does not include an EGFR-directed therapeutic agent if the tumor is both AREG LOW and EREG LOW, wherein the tumor is AREG LOW if the tumor has been histochemically or cytochemically determined to have a percentage of AREG+ tumor cells that is less than a first pre-determined cut off and wherein the tumor is considered EREG LOW if the tumor has been histochemically or cytochemically determined to have a percentage of EREG+ tumor cells that is less than a second pre-determined cut off.

Additional Embodiment 3. A method of selecting a patient with a tumor to receive a therapeutic course that includes an EGFR-directed therapeutic agent, the method comprising:
(a) histochemically or cytochemically staining a sample of the tumor for human AREG protein;
(b) histochemically or cytochemically staining a sample of the tumor for human EREG protein;
(c) quantitating a percentage of AREG+ tumor cells in the sample of the tumor and comparing the percentage to a first pre-determined cut off; and
(d) quantitating a percentage of EREG+ tumor cells in the sample of the tumor and comparing the percentage to a second pre-determined cut off,
   wherein the patient is selected to receive the therapeutic course including the EGFR-directed therapeutic agent if either the percentage of AREG+ tumor cells is greater than or equal to the first pre-determined cut off or the percentage of EREG+ tumor cells is greater than or equal to the second pre-determined cut off.

Additional Embodiment 4. A method of selecting patients with a tumor to receive a therapy that does not include an EGFR-directed therapeutic agent is provided, the method comprising:
(a) histochemically or cytochemically staining a sample of the tumor for human AREG protein;
(b) histochemically or cytochemically staining a sample of the tumor for human EREG protein;
(c) quantitating a percentage of AREG+ tumor cells in the sample of the tumor and comparing the percentage to a first pre-determined cut off; and
(d) quantitating a percentage of EREG+ tumor cells in the sample of the tumor and comparing the percentage to a second pre-determined cut off,
   wherein the patient is selected to receive the EGFR-directed therapeutic agent if the percentage of AREG+ tumor cells is less than the first pre-determined cut off and the percentage of EREG+ tumor cells is less than the second pre-determined cut off.

Additional Embodiment 5. The method of any of Additional Embodiments 1-4, wherein the first pre-determined cut off is in the range of 20% to 50% and the second pre-determined cut off is in the range of 20% to 50%.

Additional Embodiment 6. The method of Additional Embodiment 5, wherein:

the first pre-determined cut off is 20% and the second pre-determined cut off is 20%; or

the first pre-determined cut off is 25% and the second pre-determined cut off is 25%; or

the first pre-determined cut off is 20% and the second pre-determined cut off is 30%; or

the first pre-determined cut off is 25% and the second pre-determined cut off is 33.3%; or

the first pre-determined cut off is 20% and the second pre-determined cut off is 40%; or

the first pre-determined cut off is 25% and the second pre-determined cut off is 50%.

Additional Embodiment 7. A method of treating patients with a tumor, the method comprising:
(a) administering to the patient an EGFR-directed therapeutic agent if the tumor is either AREG HIGH or EREG HIGH, wherein the tumor is considered AREG HIGH if the tumor has been histochemically or cytochemically determined to have a percentage of AREG+ tumor cells that is greater than or equal to a first pre-determined positive cut off and wherein the tumor is considered EREG HIGH if the tumor has been histochemically or cytochemically determined to have a percentage of EREG+ tumor cells that is greater than or equal to a second pre-determined positive cut off; and
(b) administering to the patient a therapy course that does not include an EGFR-directed therapeutic agent if the tumor is both AREG LOW and EREG LOW, wherein the tumor is considered AREG LOW if the tumor has been histochemically or cytochemically determined to have a percentage of AREG+ tumor cells that is less than a first pre-determined negative cut off and wherein the tumor is considered EREG LOW if the tumor has been histochemically or cytochemically determined to have a percentage of EREG+ tumor cells that is less than a second pre-determined negative cut off.

Additional Embodiment 8. A method of selecting a treatment for a patient with a tumor, the method comprising:
(a) histochemically or cytochemically staining a sample of the tumor for human AREG protein;
(b) histochemically or cytochemically staining a sample of the tumor for human EREG protein;
(c) quantitating a percentage of AREG+ tumor cells in the sample of the tumor and comparing the percentage to a first pre-determined positive cut off and a first pre-determined negative cut off;
(d) quantitating a percentage of EREG+ tumor cells in the sample of the tumor and comparing the percentage to a second pre-determined positive cut off and a second pre-determined negative cut off,
(e) selecting the patient to receive a treatment course that includes an EGFR-directed therapeutic agent if the tumor is either AREG HIGH or EREG HIGH, wherein the tumor is considered AREG HIGH if the percentage of AREG+ tumor cells that is greater than or equal to the first pre-determined positive cut off and wherein the tumor is considered EREG HIGH if the percentage of EREG+ tumor cells is greater than or equal to the second pre-determined positive cut off; and
(f) selecting the patient to receive a treatment course that does not include an EGFR-directed therapeutic agent if the tumor is both AREG LOW and EREG LOW, wherein the tumor is considered AREG LOW if the percentage of AREG+ tumor cells that is less than the first pre-determined negative cut off and wherein the tumor is considered EREG LOW if the percentage of EREG+ tumor cells is less than the second pre-determined negative cut off.

Additional Embodiment 9. The method of Additional Embodiment 7 or Additional Embodiment 8, wherein:
the first pre-determined positive cut off is in the range of 30% to 50% and the first pre-determined negative cut off is in the range of 20% to 30%; and/or
the second pre-determined positive cut off is in the range of 30% to 50% and the second pre-determined negative cut off is in the range of 20% to 30%.

Additional Embodiment 10. The method of Additional Embodiment 9, wherein
the first pre-determined positive cut off is 50%;
the first pre-determined negative cut off is 20%;
the second pre-determined positive cut off is 50%; and
the second pre-determined negative cut off is 20%.

Additional Embodiment 11. The method of any of Additional Embodiments 1-10, wherein the tumor is a colorectal tumor.

Additional Embodiment 12. The method of Additional Embodiment 11, wherein the colorectal tumor is a left-sided tumor.

Additional Embodiment 13. The method of Additional Embodiment 11, wherein the colorectal tumor is a right-sided tumor.

Additional Embodiment 14. The method of any of Additional Embodiments 11-13, wherein the sample is derived from a resection of the colorectal tumor.

Additional Embodiment 15. The method of any of Additional Embodiments 11-13, wherein the sample is a biopsy sample of the colorectal tumor.

Additional Embodiment 16. The method of any of the foregoing Additional Embodiments, wherein the EGFR-directed therapeutic agent of the foregoing methods is an anti-EGFR monoclonal antibody.

Additional Embodiment 17. The method of Additional Embodiment 16, wherein the anti-EGFR monoclonal antibody is cetuximab or panitumumab.

Additional Embodiment 18. The method of any of the foregoing Additional Embodiments, wherein the treatment course comprises administering to the patient a chemotherapy

Additional Embodiment 19. The method of Additional Embodiment 18, wherein the chemotherapy comprises irinotecan.

Additional Embodiment 20. The method of any of the foregoing Additional Embodiments, wherein the tumor does not comprise a detectable amount of a mutation that confers resistance to EGFR monoclonal antibody therapy.

Additional Embodiment 21. The method of any of the foregoing Additional Embodiments, wherein the tumor is RAS wild type (RAS-wt).

Additional Embodiment 22. The method of any of the foregoing Additional Embodiments, wherein the sample of the tumor is a formalin-fixed paraffin-embedded tissue section.

Additional Embodiment 23. The method of any of the foregoing Additional Embodiments, wherein the percentage of tumor cells expressing EREG and the percentage of tumor cells expressing AREG are quantitated by an automated method.

1. A method of selecting patients with a tumor to receive a therapy that does not include an EGFR-directed therapeutic agent, the method comprising:
(a) histochemically or cytochemically staining a sample of the tumor for human AREG protein;
(b) histochemically or cytochemically staining a sample of the tumor for human EREG protein;
(c) quantitating a percentage of AREG+ tumor cells in the sample of the tumor and comparing the percentage to a first pre-determined cut off; and
(d) quantitating a percentage of EREG+ tumor cells in the sample of the tumor and comparing the percentage to a second pre-determined cut off,
wherein the patient is selected to receive the EGFR-directed therapeutic agent if the percentage of AREG+ tumor cells is less than the first pre-determined cut off and the percentage of EREG+ tumor cells is less than the second pre-determined cut off.

2. The method of claim 1, wherein the first pre-determined cut off is in the range of 20% to 50% and the second pre-determined cut off is in the range of 20% to 50%.

3. The method of claim 2, wherein: the first pre-determined cut off is 20% and the second pre-determined cut off is 20%; or the first pre-determined cut off is 25% and the second pre-determined cut off is 25%; or the first pre-determined cut off is 20% and the second pre-determined cut off is 30%; or the first pre-determined cut off is 25% and the second pre-determined cut off is 33.3%; or the first pre-determined cut off is 20% and the second pre-determined cut off is 40%; or the first pre-determined cut off is 25% and the second pre-determined cut off is 50%.

4. The method of claim 1, wherein the tumor is a colorectal tumor.

5. The method of claim 1, wherein the colorectal tumor is a left-sided tumor.

6. The method of claim 1, wherein the colorectal tumor is a right-sided tumor.

7. The method of claim 1, wherein the EGFR-directed therapeutic agent is an anti-EGFR monoclonal antibody.

8. The method of claim 7, wherein the anti-EGFR monoclonal antibody is cetuximab or panitumumab.

9. The method of claim 1, wherein the treatment course comprises administering to the patient a chemotherapy.

10. The method of claim 9, wherein the chemotherapy comprises irinotecan.

11. The method of claim 1, wherein the tumor does not comprise a detectable amount of a mutation that confers resistance to EGFR monoclonal antibody therapy.

12. The method of claim 1, wherein the tumor is RAS wild type (RAS-wt).

13. The method of claim 1, wherein the sample of the tumor is a formalin-fixed paraffin-embedded tissue section.

14. A method of treating a patient with a tumor, the method comprising:
(a) administering to the patient an EGFR-directed therapeutic agent if the tumor is either AREG HIGH or EREG HIGH, wherein the tumor is considered AREG HIGH if the tumor has been histochemically or cytochemically determined to have a percentage of AREG+ tumor cells that is greater than or equal to a first pre-determined positive cut off and wherein the tumor is considered EREG HIGH if the tumor has been histochemically or cytochemically determined to have a percentage of EREG+ tumor cells that is greater than or equal to a second pre-determined positive cut off; and
(b) administering to the patient a therapy course that does not include an EGFR-directed therapeutic agent if the tumor is both AREG LOW and EREG LOW, wherein the tumor is considered AREG LOW if the tumor has been histochemically or cytochemically determined to have a percentage of AREG+ tumor cells that is less than a first pre-determined negative cut off and wherein the tumor is considered EREG LOW if the tumor has been histochemically or cytochemically determined to have a percentage of EREG+ tumor cells that is less than a second pre-determined negative cut off.

15. The method of claim 14, wherein: the first pre-determined positive cut off is in the range of 30% to 50% and the first pre-determined negative cut off is in the range of 20% to 30%; and/or the second pre-determined positive cut off is in the range of 30% to 50% and the second pre-determined negative cut off is in the range of 20% to 30%.

16. The method of claim 15, wherein the first pre-determined positive cut off is 50%; the first pre-determined negative cut off is 20%; the second pre-determined positive cut off is 50%; and the second pre-determined negative cut off is 20%.

17. The method of claim 14, wherein the tumor is a colorectal tumor and wherein the EGFR-directed therapeutic agent is an anti-EGFR monoclonal antibody.

18. A method of selecting a treatment for a patient with a tumor, the method comprising:
(a) histochemically or cytochemically staining a sample of the tumor for human AREG protein;
(b) histochemically or cytochemically staining a sample of the tumor for human EREG protein;
(c) quantitating a percentage of AREG+ tumor cells in the sample of the tumor and comparing the percentage to a first pre-determined positive cut off and a first pre-determined negative cut off;
(d) quantitating a percentage of EREG+ tumor cells in the sample of the tumor and comparing the percentage to a second pre-determined positive cut off and a second pre-determined negative cut off,
(e) selecting the patient to receive a treatment course that includes an EGFR-directed therapeutic agent if the tumor is either AREG HIGH or EREG HIGH, wherein the tumor is considered AREG HIGH if the percentage of AREG+ tumor cells that is greater than or equal to the first pre-determined positive cut off and wherein the tumor is considered EREG HIGH if the percentage of EREG+ tumor cells is greater than or equal to the second pre-determined positive cut off; and
(f) selecting the patient to receive a treatment course that does not include an EGFR-directed therapeutic agent if the tumor is both AREG LOW and EREG LOW, wherein the tumor is considered AREG LOW if the percentage of AREG+ tumor cells that is less than the first pre-determined negative cut off and wherein the tumor is considered EREG LOW if the percentage of EREG+ tumor cells is less than the second pre-determined negative cut off.

19. The method of claim 18, wherein: the first pre-determined positive cut off is in the range of 30% to 50% and the first pre-determined negative cut off is in the range of 20% to 30%; and/or the second pre-determined positive cut off is in the range of 30% to 50% and the second pre-determined negative cut off is in the range of 20% to 30%.

20. The method of claim 18, wherein the EGFR-directed therapeutic agent is an anti-EGFR monoclonal antibody.
